(19) United States
(12) Patent Application Publication
(10) Pub. No.: US 2016/0101003 A1
(43) Pub. Date: Apr. 14, 2016

(54) ABSORBENT ARTICLE WITH FASTENING SYSTEM

(71) Applicant: The Procter & Gamble Company, Cincinnati, OH (US)

(72) Inventors: Marc Jennewein, Taunusstein (DE); Joerg Mueller, Karben (DE); Mark James Kline, Okeana, OH (US); Gabriele Elisabeth Krallmann, Frankfurt (DE); Nayda Liz Ramos-Medina, Cincinnati, OH (US); Han XU, Cincinnati, OH (US); Fang Liu, Mason, OH (US)

(21) Appl. No.: 14/878,518

(22) Filed: Oct. 8, 2015

Related U.S. Application Data
(60) Provisional application No. 62/063,445, filed on Oct. 14, 2014.

(51) Int. Cl. A61F 13/62; A61F 13/515; A61F 13/514; A61F 13/56; A61F 13/49; A61F 13/511

(52) U.S. Cl. CPC A61F 13/625; A61F 13/4906; A61F 13/511; A61F 13/51401; A61F 13/627; A61F 13/5622; A61F 13/515; A61F 2013/51452

(57) ABSTRACT

An absorbent article includes a chassis having a topsheet, a backsheet including an outer cover non-woven material, and an absorbent core disposed between the topsheet and the backsheet; at least one elastically elongatable panel joined to the chassis; and a fastening system, the fastening system having a male fastener and a female fastening material.

### FIELD

The present disclosure is generally related to disposable absorbent articles, and is more specifically related to disposable absorbent articles with improved fastening systems, aesthetics and manufacturing process reliability.

### BACKGROUND

It is known to use fastening systems to secure the corners of disposable absorbent articles, such as diapers and adult incontinence articles. Such fastening systems are used to provide a secure means for keeping such articles on the wearer during use. When such fastening systems are refastenable, adjustments may be made during use to reposition the articles, to allow a caregiver to check for soiling, and to provide a secure means for keeping such articles and their soiled contents wrapped up after use until disposal.

A traditional fastening system for use with disposable absorbent articles has a male fastener and a female fastening material. The male fastener may be disposed directly or indirectly upon the longitudinal edge of the chassis in either the front or back of the absorbent article waist regions. In use, the male fastener may be secured to the female fastening material, which is disposed upon the correspondingly opposite body portion of the front or back of the absorbent article. An exemplary fastener system that is refastenable may be provided with hooks on the male fastener that releasably engage loops on the female fastening material (i.e., a landing zone member), or vice versa. To improve fit, the end of the male fastener that does not engage the loops of the female fastening material, may be attached to one end of an elastically elongatable panel (i.e., a stretch back ear), and the other end of the elastically elongatable panel may be secured to the longitudinal edge of the chassis of the disposable absorbent article.

Such female fastening materials often take the form of an attached separate landing member that is disposed centrally on the front of a disposable absorbent article. In certain embodiments, the female fastening material can disrupt (e.g., be placed over) the artwork on the front of the diaper. Accordingly, a disposable absorbent article that includes a suitable fastening system that does not disrupt the artwork on the front of the article is of continued interest.

Further, manufacturers of disposable absorbent articles typically employ mechanical assembly lines in which a variety of absorbent article components are fed into a linear process whereby each step builds upon the last one. Such assembly lines are often referred to as “converters.” As the partial absorbent article progresses towards the end of the converter, it becomes more complete, until at the end of the line, all the necessary parts have been provided and arranged as needed to yield a completely assembled disposable absorbent article. The process is typically automated and may be controlled by various computer programs and/or human operators, as desired. In order to make the manufacture of absorbent articles economically viable, such converting lines must be capable of operating at high speeds with good repeatability and consistency between consecutive articles. Two elements that impact economic viability are 1) the nature of the disposable absorbent article components and how they will be attached to other components to form a completely assembled absorbent article, and 2) the material cost of each absorbent article component. Accordingly, a disposable absorbent article that includes a suitable fastening system and can be manufactured more economically and reliably is of continued interest.

Further, if the fastening system is refastenable, after the male and female fasteners engage and disengage multiple times, the fibers that make up the female fastener may start to disintegrate and form fuzz on the female fastener surface that engages the male fastener. Accordingly, a disposable absorbent article that includes a suitable fastening system that has a female fastener that does not significantly disintegrate after multiple engagements and disengagements with the male fastener is of continued interest.

Disposable absorbent articles with various fastening systems are known in the art. And although fastening systems with integral landing zone members (e.g., the landing zone member is formed from the backsheet material) are known, it is of continued interest to be able to obtain a disposable absorbent article with an integral landing zone member that overcomes the issues detailed above, while also achieving the desired article performance (e.g., secure engagement of fasteners in use, proper article fit, etc.).

### SUMMARY

In one embodiment, an absorbent article includes a chassis having a topsheet, a backsheet including an outer cover non-woven material, and an absorbent core disposed between the topsheet and the backsheet; and a fastening system, the fastening system having a male fastener and a female fastening material, wherein the male fastener is joined to the at least one elastically elongatable panel, and the male fastener includes a plurality of bidirectional micro-sized hooks, wherein each of the hooks has an overall hook height that is greater than or equal to one third of the overall thickness of the female fastening material but less than or equal to twice the overall thickness of the female fastening material; each of the hooks includes a cap, and each of the caps has a vertical engagement effective area that has an overall shape with at least two sides that are substantially parallel, has a width-to-thickness aspect ratio that is greater than or equal to 1 but less than or equal to 2, and is greater than or equal to 40,000 square micrometers but less than or equal to 120,000 square micrometers; and each of the caps has an overall cap height that is less than or equal to one third of the thickness of the overall thickness of the female fastening material, and less than or equal to 120 micrometers; and each of the hooks has two arms, at least one of the arms having an underside, the underside including a recessed portion, the recessed portion having a deepest recessed depth that is greater than or equal to 40% of the overall fiber cross-sectional dimension and less than or equal to 200% of the overall fiber cross-sectional dimension; and wherein the female fastening material is integral with the outer cover non-woven and the female fastening material comprises a section of a nonwoven web forming a surface approximating a plane, a machine direction and a cross direction perpendicular to the machine direction, the nonwoven web formed predominately of polymeric fibers and comprising a series of one or more consolidating bonds impressed on the surface, the one or more consolidating bonds having at least one bond shape; wherein the series is repeated to form a pattern of consolidating bonds; wherein the series is repeated in at least four rows extending predominately in the cross direction, and the series is repeated in at least four columns extending predominately in the machine direction; and wherein the bond shape has a perimeter with a greatest measurable length and a greatest measurable width, and the perimeter has a convex portion; has an aspect ratio of the greatest measurable length to the greatest measurable width of at least 2.5; and is oriented such that a line intersecting the perimeter along which the greatest measurable length exists intersects an axis lying on the surface along the machine direction to form an angle of between 1 degree and 40 degrees.

### BRIEF DESCRIPTION OF THE DRAWINGS

The above-mentioned and other features and advantages of the present disclosure, and the manner of attaining them, will become more apparent and the disclosure itself will be better understood by reference to the following description of non-limiting forms of the disclosure taken in conjunction with the accompanying drawings, wherein:

FIG. 1 is a top view of an absorbent article, wearer-facing surface facing the viewer, with some layers partially removed in accordance with the present disclosure;

FIG. 2 is a cross-sectional view of the absorbent article taken about line 2-2 of FIG. 1 in accordance with the present disclosure;

FIG. 3 is a cross-sectional view of the absorbent article taken about line 2-2 of FIG. 2 where the absorbent article has been loaded with fluid in accordance with the present disclosure;

FIG. 4 is a top view of another absorbent article, wearer-facing surface facing the viewer, with some layers partially removed in accordance with the present disclosure;

FIG. 5 is a cross-sectional view of the absorbent article taken about line 5-5 of FIG. 4 in accordance with the present disclosure;

FIG. 6 is a top view of an absorbent core of the absorbent article of FIG. 4 with some layers partially removed in accordance the present disclosure;

FIG. 7 is a cross-sectional view of the absorbent core taken about line 7-7 of FIG. 6 in accordance with the present disclosure;

FIG. 8 is a cross-sectional view of the absorbent core taken about line 8-8 of FIG. 6 in accordance with the present disclosure;

FIG. 9A is a view of a pattern of bonding surface shapes of bonding protrusions that may be imparted to the surface of a calender roller, to create another corresponding pattern of consolidating bond impressions having bond shapes in a nonwoven web;

FIG. 9B is a magnified view of the pattern of bonding surface shapes of bonding protrusions or consolidating bond impressions having bond shapes appearing in FIG. 9A;

FIG. 9C is a magnified view of the pattern of bonding surface shapes of bonding protrusions or consolidating bond impressions having bonding impressions appearing in FIG. 9A;

FIG. 10A is a view of another pattern of bonding imparted to the surface of nonwoven created by another corresponding pattern of consolidating bond protrusions on a calendar roller surface;

FIG. 10B is a magnified view of the pattern of bonding surface shapes of bonding protrusions or consolidating bond impressions on a calendar roller surface having bond shapes appearing in FIG. 10A;

FIG. 11A is an enlarged side view of a portion of a female fastening material;

FIG. 11B is an enlarged cross-sectional view of a fiber from the female fastening material of FIG. 11A;

FIG. 12 is an enlarged isometric view of a portion of a male fastener;

FIG. 13A is an enlarged view of a front of a bidirectional micro-sized hook disposed on a top surface of a substrate of a male fastener;

FIG. 13B is an enlarged side view of the hook of FIG. 13A disposed on the top surface of the substrate;

FIG. 13C is an enlarged top view of the hook of FIG. 13A;

FIG. 13D is a further enlarged view of a portion of a front of the hook of FIG. 13A;

FIG. 14 illustrates the labeling of the female fastening material from the VPT-S Method;

FIG. 15 illustrates the base plate with the female fastening material attached from the VPT-S Method;

FIG. 16 illustrates the 250 g weighted plate from the VPT-S Method;

FIG. 17 illustrates the upper plate with centering mark from the VPT-S Method;

FIG. 18 illustrates the upper plate with the male fastener attached and centered from the VPT-S Method;

FIG. 19 illustrates the upper plate orientation from the VPT-S Method;

FIG. 20 illustrates the actuator arm in the highest position from the VPT-S Method;

FIG. 21 illustrates the actuator arm in the lowest position from the VPT-S Method;

FIG. 22 is an example of a contour plot generated from JMP software;

FIG. 23 is a contour plot generated from JMP software;

FIG. 24 is a contour plot generated from JMP software;

FIG. 25 is a bond pattern for SB2;

FIG. 26 is a contour plot generated from JMP software;

FIG. 27 is a contour plot generated from JMP software;

FIG. 28 is a bond pattern known in the industry as U2888;

FIG. 29 is a contour plot generated from JMP software;

FIG. 30 is a contour plot generated from JMP software; and

FIG. 31 is a contour plot generated from JMP software.

### DETAILED DESCRIPTION

Various non-limiting embodiments will now be described to provide an overall understanding of the principles of the structure, function, manufacture, and use of the disposable absorbent articles disclosed herein. One or more examples of these non-limiting embodiments are illustrated in the accompanying drawings. Those of ordinary skill in the art will understand that the disposable absorbent articles described herein and illustrated in the accompanying drawings are non-limiting example forms and that the scope of the various non-limiting forms of the present disclosure are defined solely by the claims. The features illustrated or described in connection with one non-limiting form may be combined with the features of other non-limiting forms. Such modifications and variations are intended to be included within the scope of the present disclosure.

### INTRODUCTION

As used herein, the term “absorbent article” refers to disposable devices such as infant, child, or adult diapers, adult incontinence products, training pants, sanitary napkins, and the like which are placed against or in proximity to a body of a wearer to absorb and contain the various fluids (urine, menses, and/or runny BM) or bodily exudates (generally solid BM) discharged from the body. Typically, these absorbent articles comprise a topsheet, backsheet, an absorbent core, optionally an acquisition system and/or a distribution system (which may be comprised of one or several layers), and typically other components, with the absorbent core normally placed at least partially between the backsheet and the acquisition and/or distribution system or between the topsheet and the backsheet. Nothing in this description should be, however, considered limiting the scope of the claims. As such the present disclosure applies to any suitable form of absorbent articles (e.g., diapers, training pants, adult incontinence products, sanitary napkins).

As used herein, “bond area percentage” on a nonwoven web is a ratio of area occupied by bond impressions, to the total surface area of the web, expressed as a percentage, and measured according to the Bond Area Percentage Method set forth herein.

As used herein, a “bond impression” in a nonwoven web is the surface structure created by the impression of a bonding protrusion on a calender roller into a nonwoven web. A bond impression is a location of deformed, intermeshed or entangled, and melted or thermally fused, materials from fibers superimposed and compressed in a z-direction beneath the bonding protrusion, which form a bond. The individual bonds may be connected in the nonwoven structure by loose fibres between them. The shape and size of the bond impression approximately corresponds to the shape and size of the bonding surface of a bonding protrusion on the calender roller.

As used herein, a “column” of bonds on a nonwoven web is a group of nearest neighboring bonds of like shape and rotational orientation that are arranged along the line that extends most predominately in the machine direction.

As used herein, the term “nonwoven web” means a manufactured sheet, web, or batt of directionally or randomly orientated fibers, bonded by friction, and/or cohesion, and/or adhesion, excluding paper and products which are woven, knitted, tufted, stitch-bonded incorporating binding yarns or filaments, or felted by wet-milling, whether or not additionally needled. The fibers may be of natural or man-made origin and may be staple or continuous filaments or be formed in situ. Commercially available fibers may have diameters ranging from less than about 0.001 mm to more than about 0.2 mm and may come in several different forms such as short fibers (known as staple, or chopped), continuous single fibers (filaments or monofilaments), untwisted bundles of continuous filaments (tow), and twisted bundles of continuous filaments (yam). Nonwoven webs may be formed by many processes such as meltblowing, spunbonding, solvent spinning, electrospinning, carding, and airlaying. The basis weight of nonwoven webs is usually expressed in grams per square meter (g/m² or gsm).

As used herein, “volume mass” is the ratio of basis weight and thickness and indicates the bulkiness and fluffiness of the product, which are important properties of the nonwoven web according to the invention. The lower the value, the bulkier is the web:

Volume mass [kg/m³]=basis weight [g/m²]/thickness [mm]

As used herein, “width” or a form thereof, with respect to a diaper or training pant, refers to a dimension measured along a direction parallel to the waist edges and/or perpendicular to the longitudinal axis.

As used herein, the terms “joined”, “bonded”, or “attached” encompasses configurations whereby an element is directly secured to another element by affixing the element directly to the other element, and configurations whereby an element is indirectly secured to another element by affixing the element to intermediate member(s) which in turn are affixed to the other element.

### General Description of the Absorbent Article

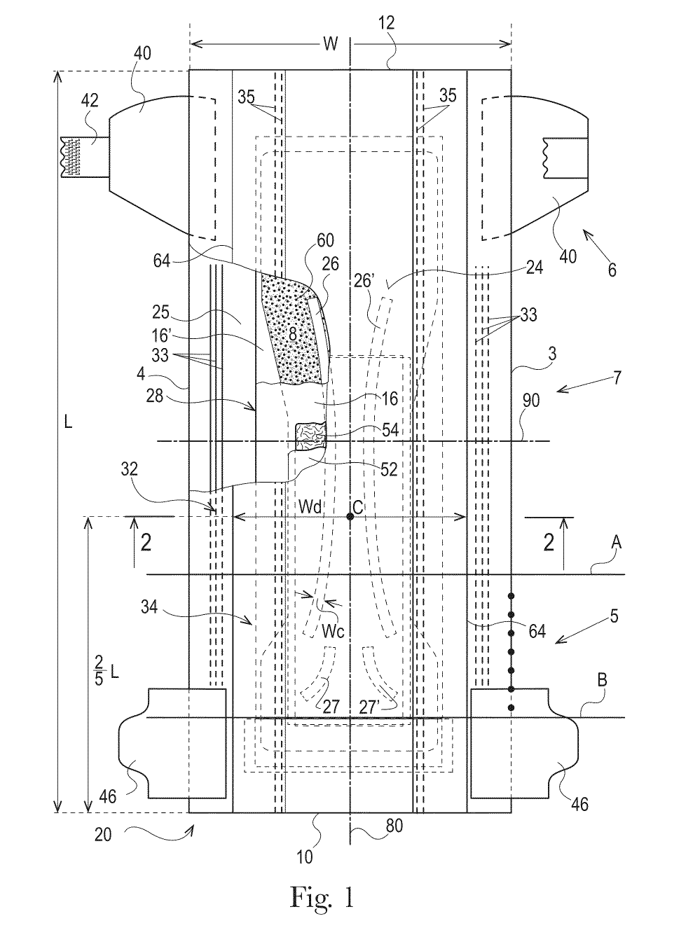

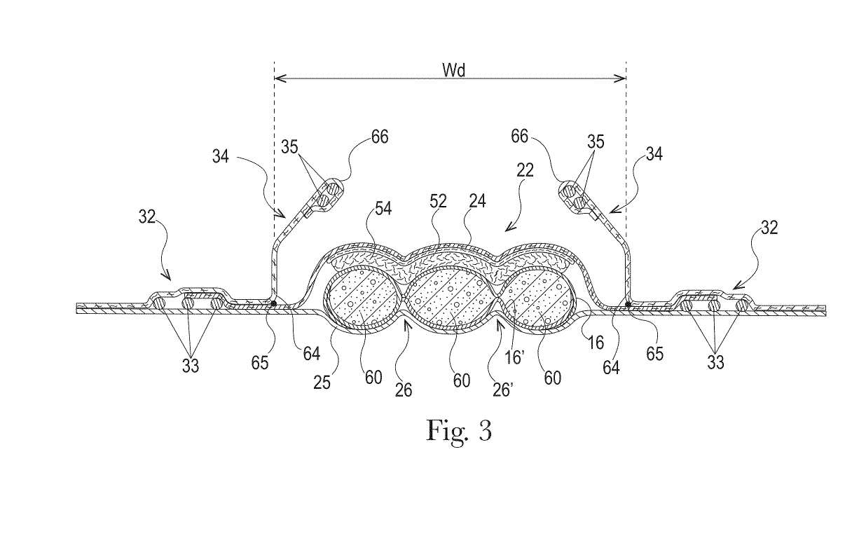

An example absorbent article in the form of a diaper 20 is represented in FIGS. 1-3. FIG. 1 is a plan view of the example diaper 20, in a flat-out state, with portions of the structure being cut-away to more clearly show the construction of the diaper 20. The wearer-facing surface of the diaper 20 of FIG. 1 is facing the viewer.

The absorbent article 20 may comprise a liquid permeable topsheet 24, a liquid impermeable backsheet 25, an absorbent core 28 positioned at least partially intermediate the topsheet 24 and the backsheet 25, and barrier leg cuffs 34. The absorbent article may also comprise an acquisition and/or distribution system (“ADS”) 50, which in the example represented comprises a distribution layer 54 and an acquisition layer 52, which will be further detailed below. The absorbent article may also comprise elasticized gasketing cuffs 32 comprising elastics 33 joined to a chassis of the absorbent article, typically via the topsheet and/or backsheet, and substantially planar with the chassis of the diaper.

The figures also show taped diaper components such as a fastening system comprising a male fastener 42 attached towards the rear edge of the article and cooperating with a portion of the backsheet 25 (the female fastening material that is integral with the outer cover non-woven of the backsheet). The absorbent article may also comprise other elements, which are not represented, such as a rear elastic waist feature, a front elastic waist feature, transverse barrier cuff(s), and/or a lotion application, for example.

The absorbent article 20 comprises a front waist edge 10, a rear waist edge 12 longitudinally opposing the front waist edge 10, a first side edge 3, and a second side edge 4 laterally opposing the first side edge 3. The front waist edge 10 is the edge of the article which is intended to be placed towards the front of the user when worn, and the rear waist edge 12 is the opposite edge. The absorbent article may have a longitudinal axis 80 extending from the lateral midpoint of the front waist edge 10 to a lateral midpoint of the rear waist edge 12 of the article and dividing the article in two substantially symmetrical halves relative to the longitudinal axis 80, with the article placed flat and viewed from above as in FIG. 1. The absorbent article may also have a lateral axis 90 extending from the longitudinal midpoint of the first side edge 3 to the longitudinal midpoint of the second side edge 4. The length, L, of the article may be measured along the longitudinal axis 80 from the front waist edge 10 to the rear waist edge 12. The width, W, of the article may be measured along the lateral axis 90 from the first side edge 3 to the second side edge 4. The article may comprise a crotch point C defined herein as the point placed on the longitudinal axis at a distance of two fifth (⅖) of L starting from the front edge 10 of the article 20. The article may comprise a front waist region 5, a rear waist region 6, and a crotch region 7. The front waist region 5, the rear waist region 6, and the crotch region 7 each define ⅓ of the longitudinal length, L, of the absorbent article.

The topsheet 24, the backsheet 25, the absorbent core 28, and the other article components may be assembled in a variety of configurations, in particular by gluing or heat embossing, for example. Example absorbent article configurations are described generally in U.S. Pat. No. 3,860,003, U.S. Pat. No. 5,221,274, U.S. Pat. No. 5,554,145, U.S. Pat. No. 5,569,234, U.S. Pat. No. 5,580,411, and U.S. Pat. No. 6,004,306.

The absorbent core 28 may comprise an absorbent material comprising at least 80% by weight, at least 90% by weight, at least 95% by weight, or at least 99% by weight of superabsorbent polymers and a core wrap enclosing the superabsorbent polymers. The core wrap may typically comprise two materials, substrates, or nonwoven materials 16 and 16′ for the top side and bottom side of the core. The core may comprises one or more channels, represented in FIG. 1 as the four channels 26, 26′ and 27, 27′. The channels 26, 26′, 27, and 27′ are optional features. Instead, the core may not have any channels or may have any number of channels.

These and other components of the example absorbent article will now be discussed in more details.

### Topsheet

The topsheet 24 may be the part of the absorbent article that is in contact with the wearer's skin. The topsheet 24 may be joined to the backsheet 25, the core 28 and/or any other layers as is known to those of skill in the art. Usually, the topsheet 24 and the backsheet 25 are joined directly to each other in some locations (e.g., on or close to the periphery of the absorbent article) and are indirectly joined together in other locations by directly joining them to one or more other elements of the article 20.

The topsheet 24 may be compliant, soft-feeling, and non-irritating to the wearer's skin. Further, a portion of, or all of, the topsheet 24 may be liquid permeable, permitting liquids to readily penetrate through its thickness. A suitable topsheet may be manufactured from a wide range of materials, such as porous foams, reticulated foams, apertured plastic films, or woven or nonwoven materials of natural fibers (e.g., wood or cotton fibers), synthetic fibers or filaments (e.g., polyester or polypropylene or bicomponent PE/PP fibers or mixtures thereof), or a combination of natural and synthetic fibers. If the topsheet 24 includes fibers, the fibers may be spunbond, carded, wet-laid, meltblown, hydroentangled, or otherwise processed as is known in the art. A suitable topsheet comprising a web of spunbond polypropylene (topically treated with a hydrophilic surfactant) is manufactured by Polymer Group, Inc., of Charlotte, N.C., under the designation P-10.

Any portion of the topsheet 24 may be coated with a lotion and/or a skin care composition as is generally disclosed in the art. The topsheet 24 may also comprise or be treated with antibacterial agents, some examples of which are disclosed in PCT Publication WO95/24173. Further, the topsheet 24, the backsheet 25 or any portion of the topsheet or backsheet may be embossed and/or matte finished to provide a more cloth like appearance.

The topsheet 24 may comprise one or more apertures to ease penetration of fluids therethrough. The size of at least the primary apertures is important in achieving the desired fluid encapsulation performance. If the primary apertures are too small, the fluids may not pass through the apertures, either due to poor alignment of the fluid source and the aperture location or due to runny fecal masses, for example, having a diameter greater than the apertures. If the apertures are too large, the area of skin that may be contaminated by “rewet” from the article is increased. Typically, the total area of the apertures at the surface of a diaper may have an area of between about 10 cm² and about 50 cm² or between about 15 cm² and 35 cm². Examples of apertured topsheets are disclosed in U.S. Pat. No. 6,632,504, assigned to BBA NONWOVENS SIMPSONVILLE. Typical diaper topsheets have a basis weight of from about 10 gsm to about 50 gsm or from about 12 gsm to about 30 gsm, but other basis weights are within the scope of the present disclosure.

### Backsheet

The backsheet 25 is generally that portion of the absorbent article 20 positioned adjacent the garment-facing surface of the absorbent core 28 and which prevents, or at least inhibits, the fluids and bodily exudates absorbed and contained therein from soiling articles such as bed sheets and undergarments. The backsheet 25 is typically impermeable, or at least substantially impermeable, to fluids (e.g., urine). The backsheet may, for example, be or comprise a thin plastic film such as a thermoplastic film having a thickness of about 0.012 mm to about 0.051 mm. Example backsheet films include those manufactured by Tredegar Corporation, based in Richmond, Va., and sold under the trade name CPC2 film. Other suitable backsheet materials may include breathable materials which permit vapors to escape from the absorbent article 20 while still preventing, or at least inhibiting, fluids from passing through the backsheet 25. Example breathable materials may include materials such as woven webs, nonwoven webs, composite materials such as film-coated nonwoven webs, microporous films such as manufactured by Mitsui Toatsu Co., of Japan under the designation ESPOIR NO and by Tredegar Corporation of Richmond, Va., and sold under the designation EXAIRE, and monolithic films such as manufactured by Clopay Corporation, Cincinnati, Ohio under the name HYTREL blend P18-3097.

The backsheet 25 may be joined to the topsheet 24, the absorbent core 28, and/or any other element of the absorbent article 20 by any attachment methods known to those of skill in the art. Suitable attachment methods are described above with respect to methods for joining the topsheet 24 to other elements of the article 20.

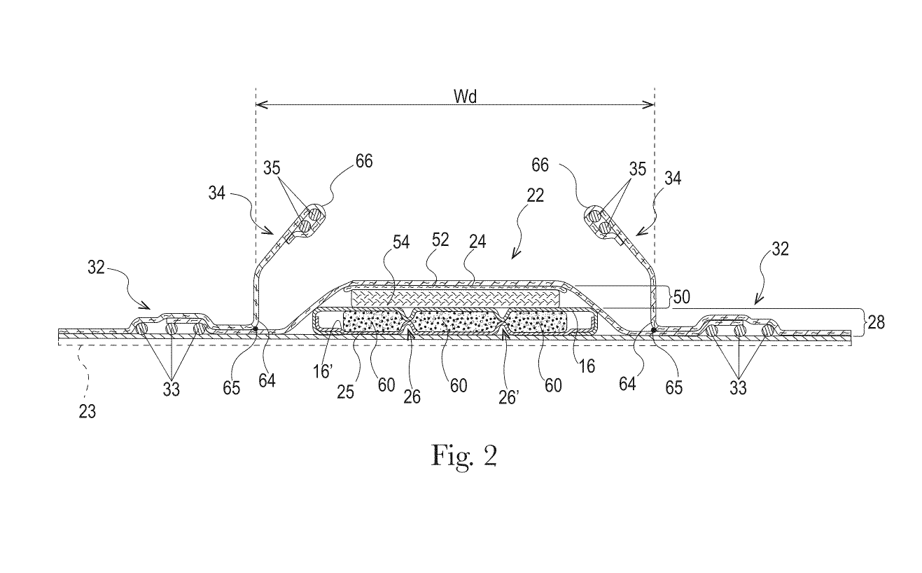

The backsheet 25 may also comprise an outer cover 23 that covers at least a portion of, or all of, the backsheet to form a soft garment-facing surface of the absorbent article. The outer cover 23 may be formed of one or more nonwoven materials. The outer cover 23 is illustrated in dash format in FIG. 2, as an example. The outer cover 23 may be joined to at least a portion of the backsheet 25 through mechanical bonding, adhesive bonding, or other suitable methods of attachment. Further, the outer cover 23 may have a bonding pattern covering the entire outer cover, or only covering a portion of the outer cover. For example, the outer cover 23 may have a bonding pattern covering the portion of the outer cover that is used as the female fastening material of the fastening system. Examples of bonding patterns that are useful on the outer cover 23 detailed herein are described in U.S. Patent Publication No. 2013/0253461A1, filed on Mar. 23, 2012.

### Bond Patterns for the Outer Cover Non-Woven of the Backsheet:

It is believed that the features of consolidating bond shapes and patterns described below may be particularly useful in applications of outer cover nonwovens of relatively low basis weights in some applications, in that it is believed that such features provide a way to enhance loft while reducing, or at least without adding, basis weight. Accordingly, for such applications, a nonwoven having a basis weight from 6.0 to 50 gsm, more preferably from 8.0 to 35 gsm, even more preferably from 9.0 to 25 gsm, and still more preferably from 10 to 20 gsm may be used.

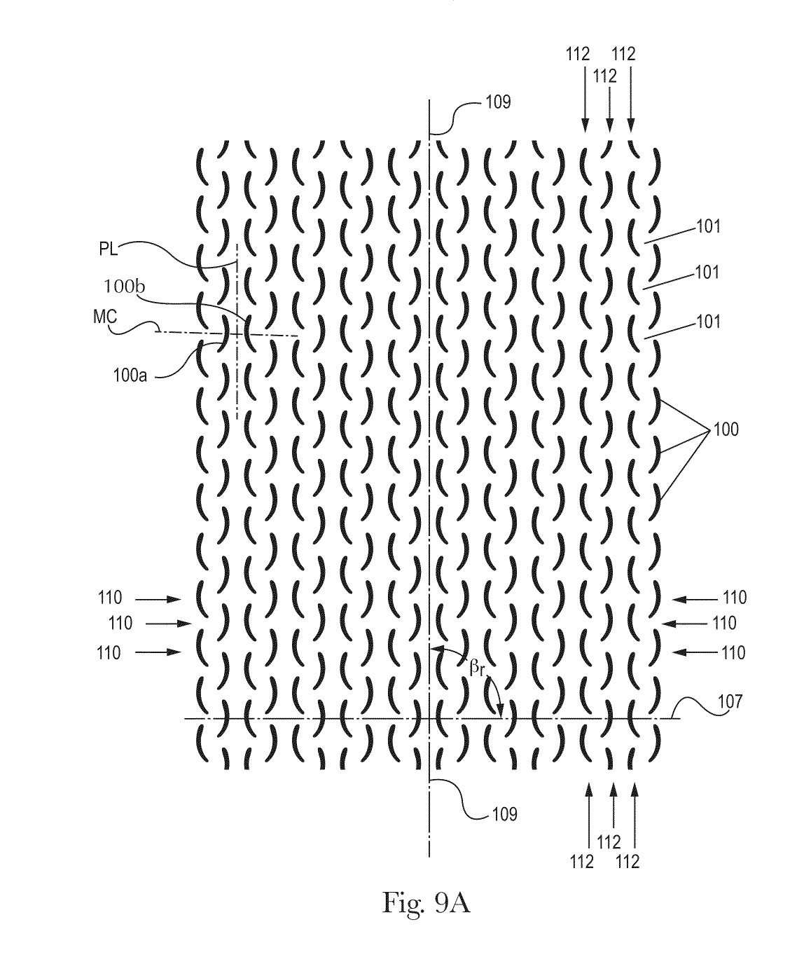

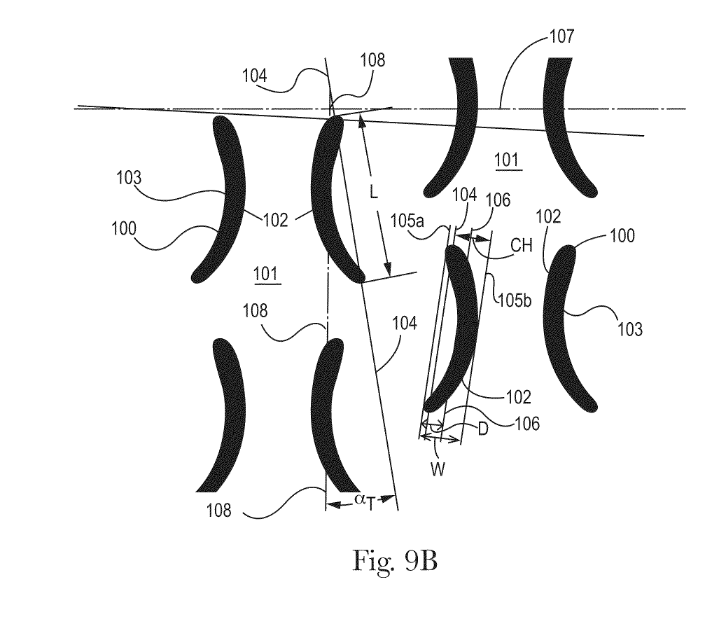

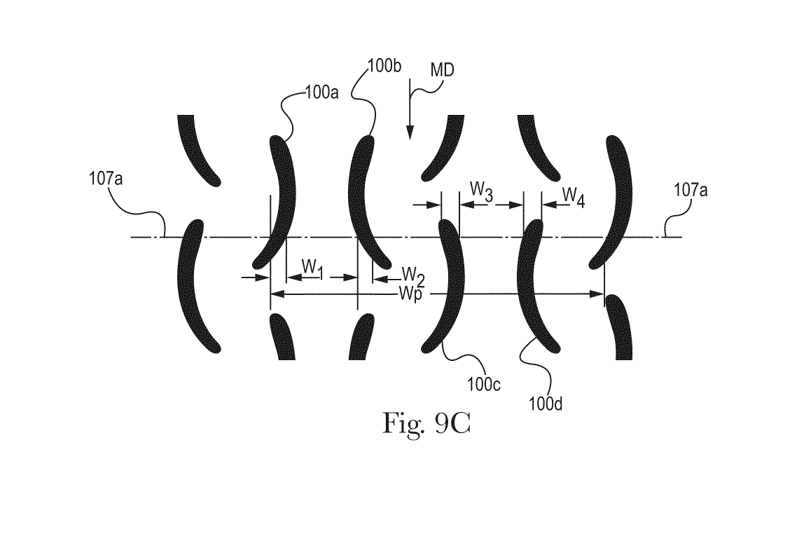

FIGS. 9A, 9B and 9C depict one example of a bonding pattern and bonding shapes that will be reflected in bond shapes of bond impressions in a nonwoven web. Bonding shapes 100 represent the shapes of bonding surfaces of bonding protrusions that may be imparted to a bonding roller by etching, machining or other methods. Such bonding protrusions on a bonding roller will impress bond impressions into a web, of like bond shapes, arranged in a like bonding pattern. Without intending to be bound by theory, it is believed that certain aspects and features of the depicted shapes and pattern may have the beneficial effect described above.

Referring to FIG. 9B, the bonding shape 100 has a greatest measurable length L, which is measured by identifying a shape length line 104 intersecting the perimeter of the shape at points of intersection that are the greatest distance apart that may be identified on the perimeter, i.e., the distance between the two farthest-most points on the perimeter. The bonding shape 100 has a greatest measurable width W which is measured by identifying respective shape width lines 105a, 105b which are parallel to shape length line 104 and tangent to the shape perimeter at one or more outermost points that are most distant from shape length line 104 on either side of it, as reflected in FIG. 9B. It will be appreciated that, for some shapes (e.g., a semicircle), one of shape width lines 105a, 105b may be coincident/colinear with shape length line 104. Greatest measurable width W is the distance between shape width lines 105a, 105b. Shapes within the scope of the present invention have an aspect ratio of greatest measurable length L to greatest measurable width W of at least 2.5, more preferably at least 2.7, and even more preferably at least 2.8. The bond shapes and sizes impressed on the nonwoven web will reflect and correspond with the bonding shapes 100 and sizes thereof on the roller.

Still referring to FIG. 9B, a bonding shape 100 may have a shape perimeter with a convex portion 102, lying on one side of the shape length line 104. FIG. 9B shows also that the convex portion may have a varying radius or radii. The varying radius/radii of the convex portion 102 may render the shape perimeter similar to the profile of the camber of an airfoil in cross section. Viewed another way, the cross-sectional profile of an airfoil has a convex portion and is asymmetric about any line or axis that traverses the profile, which can be identified. The convex portion 102 may have a camber height CH measured as the distance between shape length line 104 and the shape width line 105b that is tangent to the convex portion 102. It is believed that, for maximum beneficial impact on airflow, it may be desirable that the ratio between camber height CH and greatest measurable length L be 0.30 or less, more preferably 0.25 or less, but greater than zero. It is believed that a bonding protrusion having a cross section along a plane parallel the bonding surface, fitting this description, repeated and arranged in a pattern, has beneficial effects on acceleration and deceleration of air through nonwoven fibers at and about the nip. Again, the bond shapes and sizes impressed on the nonwoven web will reflect and correspond with the bonding shapes and sizes on the roller.

The shape perimeter may have a convex portion with or without a varying radius on both sides of shape length line 104, such that it has the overall contour of an airfoil with symmetrical camber, in cross section. In another alternative, the shape perimeter may have a convex portion on one side of shape length line 104 and a straight portion on or on the other side of shape length line 104, such that it has the overall contour of an airfoil/aircraft wing with asymmetrical camber, in cross section. In another alternative, the shape perimeter may have a convex portion on one side of shape length line 104 and a concave portion 103 disposed substantially opposite the concave portion, as reflected in FIG. 9B, such that it has the overall contour of an airfoil/aircraft wing with asymmetrical camber and relatively high-loft, low-speed features, in cross section.

The extent of the concavity of concave portion 103 may be quantified by measuring the depth thereof, relative the greatest measurable length. The concavity depth D may be measured by identifying a shape concavity line 106 that is parallel with the shape length line 104 and tangent to the deepest point along the concave portion 103. The concavity depth D is the distance between the shape width line 105a facing the concavity and the shape concavity line 106. The extent of the concavity of concave portion 103 may be expressed as a ratio of concavity depth D to shape length L (hereinafter, “concavity depth ratio”). Although shapes that do not have a concave portion 103 are contemplated, it may be desirable that a bonding shape has a concave portion having a concavity depth ratio between 0.00 and 0.30, more preferably between 0.00 and 0.25, and even more preferably between 0.00 and 0.20. Again, the bond shapes and sizes impressed on the nonwoven web will reflect and correspond with the bonding shapes and sizes on the roller.

Whilst the explanation above refers to bonding protrusions and resulting consolidated bond shapes in the web, which have bonding shape/bond shape perimeters following “convex” and/or “concave” (impliedly, smooth) curves, it may be appreciated that the effect may be substantially realized by approximating such smooth curves with chains of straight line segments. Accordingly, each of the terms “convex” and “concave” herein includes a portion of a shape perimeter formed of a chain of 5 or more straight line segments lying on one side of a shape length line and connected end-to-end, that is each a chord of a smooth convex or concave curve lying on one side of the shape length line, or portion of a curve lying on one side of the shape length line that does not include an inflection point.

Without intending to be bound by theory, it is believed that calender roller bonding protrusions having bonding shapes with one or more features as described above have aerodynamic effects on air flow in and about the nip, that cause acceleration and deceleration of air in and about the interstices of the nonwoven fibers in a way that repositions the fibers, and may affect teasing or fluffing, adding loft and caliper.

Additionally, the rotational orientations of the protrusions affect the orientations of the bonding protrusions at the nip, and it is believed that this has an impact. Bonding shapes 100 and the bonding protrusions supporting them may be arranged along an individual shape tilt angle relative the machine and cross directions. Without intending to be bound by theory, it is believed that the shape tilt angle should not exceed a certain amount for the bonding protrusion to have maximum beneficial effect on air flow. Referring again to FIG. 9B, the shape tilt angle α_{T} may be expressed as the smaller angle formed by the intersection of an axis along the machine direction 108 and the shape length line 104. It is believed, that the shape and the shape tilt angle have cooperating effects on the air flow. In the case of an asymmetric bonding shape, such as the described airfoil-like shape, it is believed that this asymmetric bonding shape is sufficient for effecting the desired changes in air flow. However, a rotational orientation with a tilt angle of more than zero may enhance the effect. With respect to a bonding shape that is not asymmetric, it is believed that the shape tilt angle α_{T} provides the desired effects on air flow, such that it then should not be less than 1 degree and should not exceed 40 degrees, more preferably, 30 degrees, and still more preferably, 20 degrees. It is believed that a shape tilt angle within this range effectively provides air flow through the nip, while at the same time, imparts cross-direction vector components to air flows through the nip. Conversely, a shape tilt angle greater than 40 degrees may create too much of an obstruction to air flow through the nip to have a beneficial effect, and even greater shape tilt angles combined with sufficient density of bonding protrusions may have the effect of creating enough obstruction at the nip to substantially divert airflow from the nip, i.e., toward the sides of the bonding rollers, rather than through the nip. The bond shapes and rotational orientations impressed on the nonwoven web will reflect and correspond with the bonding shapes and rotational orientations on the roller.

It is believed that air flows having cross-direction vector components flowing across or through the batt/web as it passes through and exits the nip may urge fibers in the cross-direction, helping add loft, caliper and/or cross direction tensile strength. It will be appreciated that the fibers of many nonwoven batts are laid down in the nonwoven web manufacturing process with a general machine direction orientation or bias, which tends to result in the finished web having relatively greater machine direction tensile strength, and relatively less cross direction tensile strength. Thus, any process that tends to impart some added cross-direction orientation to the fibers prior to bonding may be useful for increasing cross direction tensile strength, bringing about better balance between machine direction tensile strength and cross-direction tensile strength, and adding loft such as by repositioning of the fibers in the z-direction. It is believed that, for best results, it may be even more desirable that shape tilt angle α_{T} is between 5 degrees and 15 degrees, more preferably between 8 degrees and 12 degrees, and even more preferably between 9 degrees and 11 degrees, for the most beneficial effects on airflow at the line speeds contemplated herein. The rotational orientation of the bonding pattern impressed on the nonwoven web will reflect and correspond with the rotational orientation of the bonding pattern on the roller.

As suggested above, in order to gain the benefit of energy from a substantial mass of air flowing through the nip, it is also believed desirable that a pattern of bonding protrusions not be excessively obstructive of air flow through the nip, nor that it remove too much energy from the air flow by overly slowing, or halting, and absorbing the energy from, forward (machine-direction) momentum of air flows. Referring to FIG. 9C, a nip line 107a along the cross direction is identified along a pattern where the bonding shapes occupy the greatest proportion of distance along a cross direction line that can be identified in a pattern. Thus, nip line 107a located as shown represents a cross-direction line along which bonding protrusions presented the greatest amount of obstruction that can be identified in a particular pattern, to air flow through the nip, during the bonding process. A repeating series of shapes can be identified; in this example, the repeating series consists of the four shapes 100a, 100b, 100c and 100d. Widths w₁, w₂, w₃, and w₄ of the identified shapes 100a, 100b, 100c, 100d in the repeating series reflect restriction of air flow along the nip line 107a. Width wₚ is the width of the entire repeating series, including the distances between the bonding shapes. The proportion of maximum restriction along the nip length for the pattern is reflected by the ratio (w₁+w₂+w₃+w₄ . . . +wₙ)/wₚ, referred to herein as the nip airflow restriction ratio (where “w” is the cross-direction width along the nip line 107a of a bonding shape perimeter, and “n” is the number of bonding shapes along nip line 107a that make up a repeating series). In order that a bonding pattern allows for effective air flow through the nip in order to take advantage of energy of moving air, it may be desirable that the nip airflow restriction ratio be 0.40 or less, more preferably 0.30 or less, and even more preferably 0.25 or less. The bond shapes, rotational orientations and density/numerosity per unit surface area of bond impressions on the nonwoven web will reflect and correspond with the bonding shapes, rotational orientations and density/numerosity per unit surface area of bonding protrusions on the roller, and thus, also reflect the airflow restriction ratio.

Referring to FIGS. 10A and 10B, an alternative bonding pattern is depicted. The repeated bonding shape 100 and profile of the associated bonding protrusion is a composite of two generally convex/concave sub-shapes joined or superimposed at their respective tips, in reversed orientation, to form an open “S” shape which is rotationally symmetric about this juncture of the component sub-shapes, respectively its middle inflection point. It will be appreciated, however, that the depicted repeated “S” shape may have several of the features of the bonding shape depicted in FIGS. 9A and 9B, described above, which are believed to be beneficial. The depicted bonding shape 100 in FIGS. 10A and 10B has a greatest measurable length L and greatest measurable width W, measured with respect to shape length line 104 and shape width lines 105a, 105b, identified in the manner described above. As set forth above, bonding shapes 100 within the scope of the present invention have an aspect ratio of greatest measurable length L to greatest measurable width W of at least 2.5, more preferably at least 2.7, and even more preferably at least 2.8.

The depicted bonding shape in FIGS. 10A and 10B also has convex portions 102a, 102b along its perimeter. One or both of the convex portions 102a, 102b may have varying radii, and have camber heights CH_{A} and CH_{B}. It is believed that, for maximum beneficial impact on airflow, it may be desirable that the ratio between camber height CH and the greatest measurable length L also be 0.30 or less, more preferably 0.25 or less, but greater than zero.

The depicted bonding shape also has concave portions 103a and 103b along its perimeter. Concavity depth Da is the distance between shape width line 105a facing concavity 103a, and shape concavity line 106a. Concavity depth Db is the distance between shape width line 105b facing concavity 103b, and shape concavity line 106b. Although bonding shapes that do not have a concave portion 103a, 103b along their perimeters are contemplated, it may be desirable that a bonding shape perimeter has one or more concave portions such as concave portions 103a, 103b having a concavity depth ratio:

Concavity depth/(L*nc)≦0.30, more preferably 0.25, and even more preferably 0.20, where nc is the number of fully enclosed shapes that are defined by portions of the bonding shape perimeter and the shape length line, which evidence concavities. For example, for the “S” shape shown in FIG. 10B, nc=2 because there are 2 such fully enclosed shapes 124a and 124b.

The shapes 100 in FIGS. 10A and 10B also may have a shape tilt angle α_{T} determined as set forth above, and within the ranges set forth. The geometric features of the bond shapes and pattern on the nonwoven web will reflect and correspond with those of the shape, size, rotational orientation, density and arrangement of the bond shapes 100.

It is also believed that arranging the bonding protrusions in a pattern such that a relatively straight, unobstructed passageway between them exists along recessed areas 101 at the nip, at least partially along the machine direction, may have beneficial effects. Referring to FIGS. 9A and 10A, it can be seen that each example has a cross-nip airflow line 109 that can be identified, that intersects no bonding shape, and intersects a cross direction axis 107 at an angle such that it has a machine direction vector component. Cross-nip airflow line 109 intersects cross direction axis 107 to form a smaller angle, identified herein as cross-nip airflow angle β_{A}. It is believed that cross-nip airflow angle β_{A} is preferably greater than 45 degrees, more preferably between 50 degrees and 90 degrees, and even more preferably between 60 degrees and 90 degrees. It is believed desirable that cross-nip airflow line 109 should extend indefinitely without intersecting a bonding shape 100, but at a minimum, past at least 8 rows 110 of bonding shapes 100 without intersecting a bond shape. Again, geometric features of the bond shapes and pattern on the nonwoven web will reflect and correspond with those of the shape, size, rotational orientation, density and arrangement of the bond shapes 100.

Another aspect of the bonding shapes and patterns depicted in, e.g., FIGS. 9A-10B is that they may have any combination of the above-described aspect ratios, maximum nip airflow restriction ratio (0.40 or less), shape asymmetry, shape tilt angles, and other features, and may also reflect use of adjacent pairs of bonding protrusions that define air passageways through the nip that alternately narrow and widen, or converge and diverge, in the manner of a venturi. For example, referring again to FIGS. 9A and 10A, two adjacent bond shapes 100a, 100b may be identified. Herein, “adjacent” means that at least portions of the perimeters of a pair of shapes face each other with no intervening shapes between them; and that the pair of shapes has machine-direction overlap. The pair of shapes has machine-direction overlap if one or more cross-direction lines 107 that are tangent to and/or cross the perimeters of each of the shapes may be identified. A minimum passageway clearance line MC may be identified connecting the perimeters of the shapes 100a, 100b, at the location where the shortest measurable distance between the perimeters exists. The minimum passageway clearance line MC will necessarily meet the perimeter of each of the adjacent shapes where line MC is normal to the perimeter, and line MC identifies the point of greatest constriction of an air passageway between the shapes (i.e., through the corresponding bonding protrusions) proximate and through the nip. A passageway line PL may be identified, perpendicular to the minimum passageway clearance line MC and lying between the adjacent shapes 100a, 100b.

The minimum passageway clearance line MC crosses and identifies a “venturi passageway” if the perimeter of each of the adjacent shapes 100a, 100b diverges away from the passageway line PL moving along the perimeter away from the minimum clearance line MC in both directions. It can be seen in FIGS. 9A and 10A that adjacent shapes 100a, 100b embody this feature.

Without intending to be bound by theory, it is believed that such venturi passageways have the effect of causing localized zones of acceleration and deceleration, and increases and decreases in pressure, as well as turbulence, of air as it passes through the nip. It is believed that these effects serve to tease and/or fluff the fibers of the batt and web about the nip.

For purposes of downstream handling and manufacturing processes, it may be desirable to ensure that no line along the machine direction exists along the nonwoven web surface that is indefinitely long without intersecting a bond impression. This condition (indefinitely long machine direction strip of web without bonds) may result in relatively long lengths of unbonded fibers that may be prone to moving away from a cutting knife in downstream machine direction web slitting operations, resulting in a poorly defined or sloppy slit edge. Additionally, such long, unbonded fibers may also separate from a manufactured edge or slit edge of the web (fraying), which may cause other difficulties in downstream operations. To avoid this condition, it may be desirable to impart a pattern angle γ_{P} to the bonding pattern. Referring to FIG. 10A, pattern angle γ_{P} may be expressed as the smaller angle formed by the intersection of a line 111 connecting like points on repeating, similarly oriented shapes in columns 112, and a machine direction axis. To avoid the above-mentioned problems, it may be desirable that pattern angle γ_{P} be greater than 0 degrees. A pattern angle greater than 0 degrees will ensure that an indefinitely long machine direction strip of web without bonds will not exist. To avoid creating complications with respect to the air flow benefits of the pattern, however, it may be desirable to limit pattern angle γ_{P} to 4 degrees or less, more preferably 3 degrees or less, and even more preferably 2.5 degrees or less. Again, features of the bond pattern on the nonwoven web including pattern angle will reflect and correspond with those of the pattern and pattern angle γ_{P} on the roller.

An additional aspect that it believed important is bonding area of a roller, reflected in bond area on the web. Imagining a pattern of bonding surfaces having shapes reflected in FIGS. 9A and 10A impressed on a surface of a nonwoven web, bonding area and bond area is the area occupied by the bonding shapes on the roller and bond shapes impressed on the surface of the web. In the field of nonwoven web manufacturing, bonding area is often expressed as a percentage, calculated as:

$$Bonding Area \% = [ \frac{( bonding area within a surface area unit )}{( total surface area of the surface area unit )} ] \times 100 \%$$

The bonding area reflects the combination of bonding protrusion density (number of bonding protrusions per unit surface area) and average surface area of the bonding shapes 100 in the unit surface area. Thus, increasing the number of bonding protrusions and/or increasing the surface area of the individual bond shapes 100 increases the bonding area, and vice versa. It is believed that bonding area has an impact on the entrainment of air as well as the proportion of entrained air carried toward the nip, which will pass through the nip. If bonding area is relatively greater, this means that more and/or larger bonding protrusions are present at the nip point at any time to obstruct air flow through the nip; conversely, if bonding area is relatively less, this means that fewer and/or smaller bonding protrusions are present at the nip point at any time to obstruct air flow through the nip. Bond area has another effect as well. Increasing bond area increases the number and proportion of the fibers in the nonwoven web that are bonded together, and vice versa. Within a certain range of bond area, tensile strength of the nonwoven web in the machine and/or cross directions may be increased by increasing the bond area. However, bending stiffness of the nonwoven web may be correspondingly increased, and loft decreased—compromising the soft feel and/or appearance of the nonwoven. In order to best realize the benefits of air flow, air compression and channeling believed to be occurring through use of the bond shapes described herein, enhancing loft, while still imparting satisfactory tensile properties to the web, it is believed that bonding area should be in the range of 4.0% and 18%, more preferably between 6% and 16%, and even more preferably between about 8% and 14%. At the line speeds contemplated herein, and relative to the bonding area, the average surface area per bonding shape affects bonding area and bonding protrusion density. It is believed desirable that the average bonding shape 100 surface area be in the range of 0.3 mm² and 10 mm². Correspondingly, it is believed desirable that the density of the bonding protrusions, and correspondingly, the impressed bond shapes, be between 0.4 bonding protrusions/cm² for bonding shape/bond shape area of 10 mm² at 4% bonding area and 60 bonding protrusions/cm² for bonding shape/bond shape area of 0.3 mm² at 18% bonding area Similar calculations of bonding protrusion density and average bond shape surface area to arrive at the bond areas in the ranges set forth above, will be appreciated. The surface area and density of bond shapes impressed on the nonwoven web will reflect and correspond with those of the bonding shapes, and thus, the bond area on the web will reflect and correspond with the bonding area on the roller as well.

It is believed that surface features of a bonding roller including the bonding protrusions affect the above detailed air flows. Particularly at the nip, the profiles of bonding protrusions present obstructions to airflow, while the recessed areas between the bonding protrusions present passageways. Thus, it is believed that for certain configurations, shapes, and positions of bonding protrusions, as will be reflected in the bond impressions created in the web, rotational orientation(s) and repeating patterns of the bonding shapes can be selected and formed to have a beneficial effect on these air flows. It is believed, further, that patterns of bonding protrusions having bonding surface shapes with certain features, reflected in the bonding surfaces and the cross sections of the protrusions along planes substantially parallel with the bonding surfaces, rotational orientations relative the plane approximated by the web surface, and spacing, may be employed to channel these air flows in a way that causes them to reposition the fibers during the calender bonding process, such as by teasing or fluffing the fibers, thus providing an enhanced calender-bonded nonwoven web having greater loft/caliper than a similar nonwoven web having other consolidated bond shapes and patterns, all other variables being the same.

### Absorbent Core

As used herein, the term “absorbent core” refers to the component of the absorbent article having the most absorbent capacity and comprising an absorbent material and a core wrap or core bag enclosing the absorbent material. The term “absorbent core” does not include the acquisition and/or distribution system or any other components of the article which are not either integral part of the core wrap or core bag or placed within the core wrap or core bag. The absorbent core may comprise, consist essentially of, or consist of, a core wrap, an absorbent material (e.g., superabsorbent polymers) as discussed, and glue.

The absorbent core 28 may comprise an absorbent material with a high amount of superabsorbent polymers (herein abbreviated as “SAP”) enclosed within the core wrap. The SAP content may represent 70%-100% or at least 70%, 75%, 80%, 85%, 90%, 95%, 99%, or 100%, by weight of the absorbent material, contained in the core wrap. The core wrap is not considered as absorbent material for the purpose of assessing the percentage of SAP in the absorbent core. The core may also contain airfelt or cellulosic fibers with or without SAP.

By “absorbent material” it is meant a material which has some absorbency property or liquid retaining properties, such as SAP, cellulosic fibers as well as synthetic fibers. Typically, glues used in making absorbent cores have no or little absorbency properties and are not considered as absorbent material. The SAP content may be higher than 80%, for example at least 85%, at least 90%, at least 95%, at least 99%, and even up to and including 100% of the weight of the absorbent material contained within the core wrap. This provides a relatively thin core compared to a conventional core typically comprising between 40-60% SAP and high content of cellulose fibers. The conventional cores are also within the scope of the present disclosure. The absorbent material may in particular comprises less than 15% weight percent or less than 10% weight percent of natural, cellulosic, or synthetic fibers, less than 5% weight percent, less than 3% weight percent, less than 2% weight percent, less than 1% weight percent, or may even be substantially free of natural, cellulosic, and/or synthetic fibers.

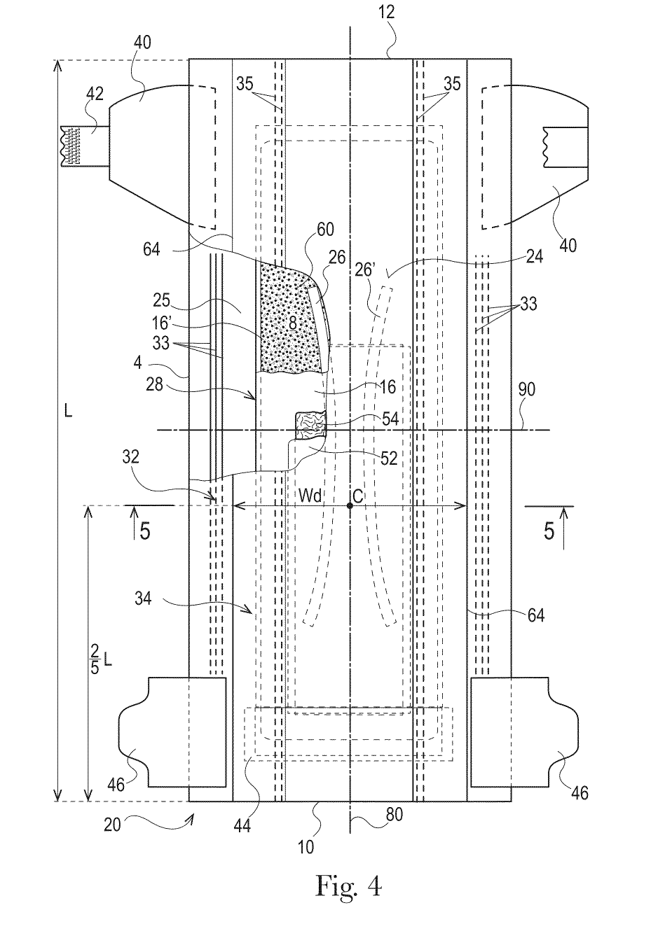

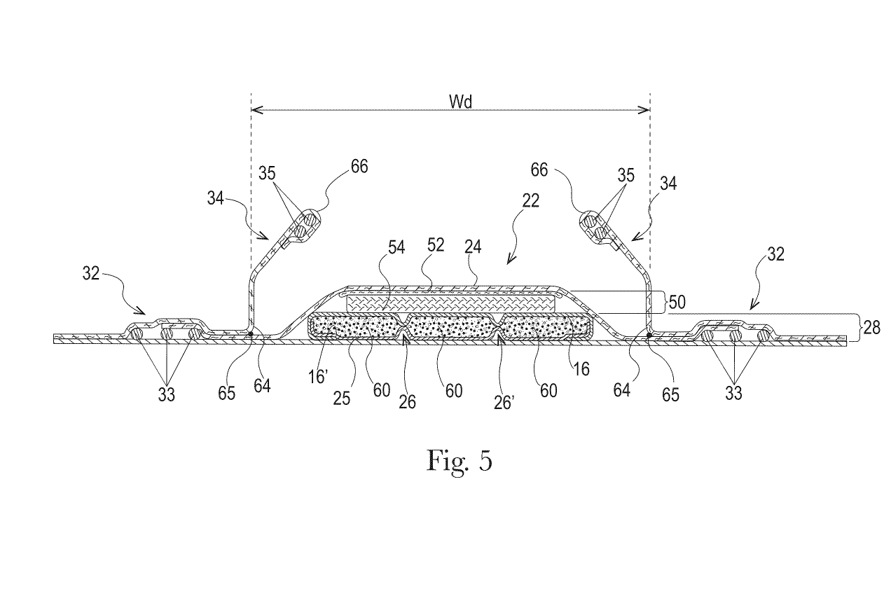

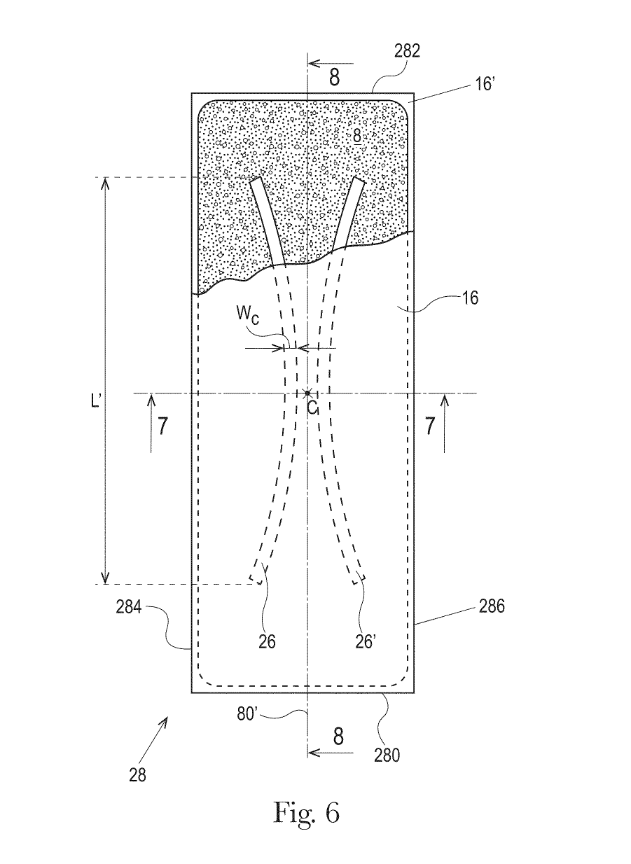

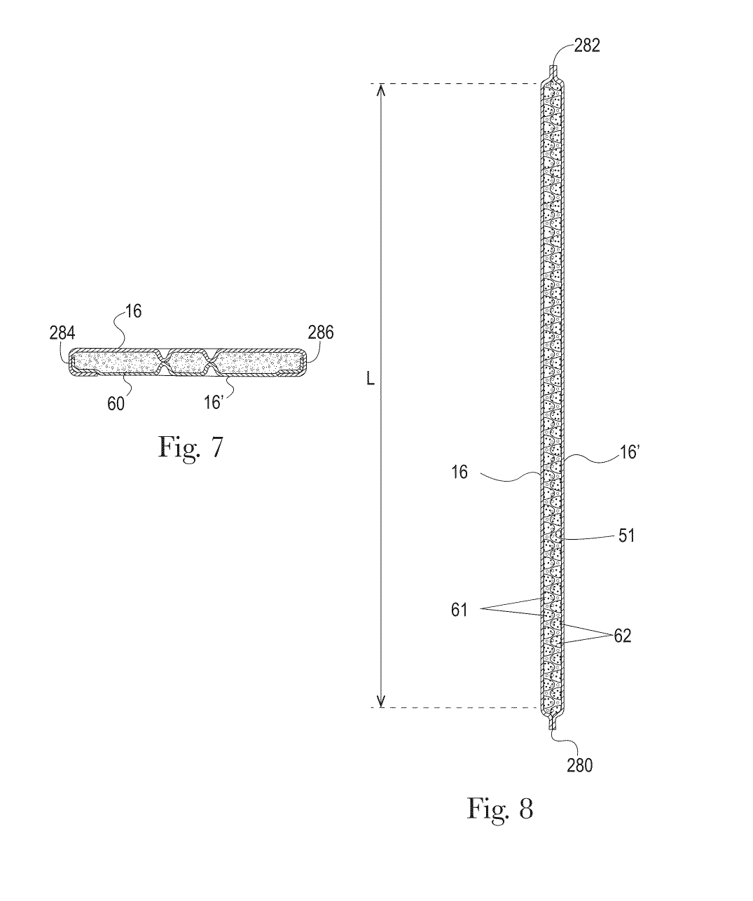

The example absorbent core 28 of the absorbent article 20 of FIGS. 4-5 is shown in isolation in FIGS. 6-8. The absorbent core 28 may comprises a front side 280, a rear side 282, and two longitudinal sides 284, 286 joining the front side 280 and the rear side 282. The absorbent core 28 may also comprise a generally planar top side and a generally planar bottom side. The front side 280 of the core is the side of the core intended to be placed towards the front waist edge 10 of the absorbent article. The core 28 may have a longitudinal axis 80′ corresponding substantially to the longitudinal axis 80 of the absorbent article 20, as seen from the top in a planar view as in FIG. 1. The absorbent material may be distributed in higher amount towards the front side 280 than towards the rear side 282 as more absorbency may be required at the front in particular absorbent articles. The front and rear sides 280 and 282 of the core may be shorter than the longitudinal sides 284 and 286 of the core. The core wrap may be formed by two nonwoven materials, substrates, laminates, or other materials, 16, 16′ which may be at least partially sealed along the sides 284, 286 of the absorbent core 28. The core wrap may be at least partially sealed along its front side 280, rear side 282, and two longitudinal sides 284, 286 so that substantially no absorbent material leaks out of the absorbent core wrap. The first material, substrate, or nonwoven 16 may at least partially surround the second material, substrate, or nonwoven 16′ to form the core wrap, as illustrated in FIG. 7. The first material 16 may surround a portion of the second material 16′ proximate to the first and second side edges 284 and 286.

The absorbent core may comprise adhesive, for example, to help immobilizing the SAP within the core wrap and/or to ensure integrity of the core wrap, in particular when the core wrap is made of two or more substrates. The adhesive may be a hot melt adhesive, supplied, by H.B. Fuller, for example. The core wrap may extend to a larger area than strictly needed for containing the absorbent material within.

Cores comprising relatively high amount of SAP with various core designs are disclosed in U.S. Pat. No. 5,599,335 (Goldman), EP 1,447,066 (Busam), WO 95/11652 (Tanzer), U.S. Pat. Publ. No. 2008/0312622A1 (Hundorf), and WO 2012/052172 (Van Malderen).

The absorbent material may be a continuous layer present within the core wrap. Alternatively, the absorbent material may be comprised of individual pockets or stripes of absorbent material enclosed within the core wrap. In the first case, the absorbent material may be, for example, obtained by the application of a single continuous layer of absorbent material. The continuous layer of absorbent material, in particular of SAP, may also be obtained by combining two absorbent layers having discontinuous absorbent material application patterns, wherein the resulting layer is substantially continuously distributed across the absorbent particulate polymer material area, as disclosed in U.S. Pat. Appl. Pub. No. 2008/0312622A1 (Hundorf), for example. The absorbent core 28 may comprise a first absorbent layer and a second absorbent layer. The first absorbent layer may comprise the first material 16 and a first layer 61 of absorbent material, which may be 100% or less of SAP. The second absorbent layer may comprise the second material 16′ and a second layer 62 of absorbent material, which may also be 100% or less of SAP. The absorbent core 28 may also comprise a fibrous thermoplastic adhesive material 51 at least partially bonding each layer of absorbent material 61, 62 to its respective material 16 or 16′. This is illustrated in FIGS. 7-8, as an example, where the first and second SAP layers have been applied as transversal stripes or “land areas” having the same width as the desired absorbent material deposition area on their respective substrate before being combined. The stripes may comprise different amounts of absorbent material (SAP) to provide a profiled basis weight along the longitudinal axis of the core 80. The first material 16 and the second material 16′ may form the core wrap.

The fibrous thermoplastic adhesive material 51 may be at least partially in contact with the absorbent material 61, 62 in the land areas and at least partially in contact with the materials 16 and 16′ in the junction areas. This imparts an essentially three-dimensional structure to the fibrous layer of thermoplastic adhesive material 51, which in itself is essentially a two-dimensional structure of relatively small thickness, as compared to the dimension in length and width directions. Thereby, the fibrous thermoplastic adhesive material may provide cavities to cover the absorbent material in the land areas, and thereby immobilizes this absorbent material, which may be 100% or less of SAP.

The thermoplastic adhesive used for the fibrous layer may have elastomeric properties, such that the web formed by the fibers on the SAP layer is able to be stretched as the SAP swell. Elastomeric, hot-melt adhesives of these types are described in more detail in U.S. Pat. No. 4,731,066 issued to Korpman on Mar. 15, 1988. The thermoplastic adhesive material may be applied as fibers.

### Superabsorbent Polymer (SAP)

“Superabsorbent polymers” (“SAP”), as used herein, refer to absorbent materials which are cross-linked polymeric materials that can absorb at least 10 times their weight of an aqueous 0.9% saline solution as measured using the Centrifuge Retention Capacity (CRC) test (EDANA method WSP 241.2-05E). The SAP used may have a CRC value of more than 20 g/g, more than 24 g/g, from 20 to 50 g/g, from 20 to 40 g/g, or from 24 to 30 g/g, specifically reciting all 0.1 g/g increments within the above-specified ranges and any ranges created therein or thereby. The SAP useful with the present disclosure may include a variety of water-insoluble, but water-swellable polymers capable of absorbing large quantities of fluids.

The superabsorbent polymer may be in particulate form so as to be flowable in the dry state. Particulate absorbent polymer materials may be made of poly(meth)acrylic acid polymers. However, starch-based particulate absorbent polymer material may also be used, as well as polyacrylamide copolymer, ethylene maleic anhydride copolymer, cross-linked carboxymethylcellulose, polyvinyl alcohol copolymers, cross-linked polyethylene oxide, and starch grafted copolymer of polyacrylonitrile.

The SAP may be of numerous shapes. The term “particles” refers to granules, fibers, flakes, spheres, powders, platelets and other shapes and forms known to persons skilled in the art of superabsorbent polymer particles. The SAP particles may be in the shape of fibers, i.e., elongated, acicular superabsorbent polymer particles. The fibers may also be in the form of a long filament that may be woven. SAP may be spherical-like particles. The absorbent core may comprise one or more types of SAP.

For most absorbent articles, liquid discharges from a wearer occur predominately in the front half of the absorbent article, in particular for a diaper. The front half of the article (as defined by the region between the front edge and a transversal line placed at a distance of half L from the front waist edge 10 or rear waist edge 12 may therefore may comprise most of the absorbent capacity of the core. Thus, at least 60% of the SAP, or at least 65%, 70%, 75%, 80%, or 85% of the SAP may be present in the front half of the absorbent article, while the remaining SAP may be disposed in the rear half of the absorbent article. Alternatively, the SAP distribution may be uniform through the core or may have other suitable distributions.

The total amount of SAP present in the absorbent core may also vary according to expected user. Diapers for newborns may require less SAP than infant, child, or adult incontinence diapers. The amount of SAP in the core may be about 5 to 60 g or from 5 to 50 g, specifically reciting all 0.1 increments within the specified ranges and any ranged formed therein or thereby. The average SAP basis weight within the (or “at least one”, if several are present) deposition area 8 of the SAP may be at least 50, 100, 200, 300, 400, 500 or more g/m². The areas of the channels (e.g., 26, 26′, 27, 27′) present in the absorbent material deposition area 8 are deduced from the absorbent material deposition area to calculate this average basis weight.

### Core Wrap

The core wrap may be made of a single substrate, material, or nonwoven folded around the absorbent material, or may comprise two (or more) substrates, materials, or nonwovens which are attached to another. Typical attachments are the so-called C-wrap and/or sandwich wrap. In a C-wrap, as illustrated, for example, in FIGS. 2 and 7, the longitudinal and/or transversal edges of one of the substrates are folded over the other substrate to form flaps. These flaps are then bonded to the external surface of the other substrate, typically by gluing.

The core wrap may be formed by any materials suitable for receiving and containing the absorbent material. Typical substrate materials used in the production of conventional cores may be used, in particular paper, tissues, films, wovens or nonwovens, or laminates or composites of any of these.

The substrates may also be air-permeable (in addition to being liquid or fluid permeable). Films useful herein may therefore comprise micro-pores.

The core wrap may be at least partially sealed along all the sides of the absorbent core so that substantially no absorbent material leaks out of the core. By “substantially no absorbent material” it is meant that less than 5%, less than 2%, less than 1%, or about 0% by weight of absorbent material escape the core wrap. The term “seal” is to be understood in a broad sense. The seal does not need to be continuous along the whole periphery of the core wrap but may be discontinuous along part or the whole of it, such as formed by a series of seal points spaced on a line. A seal may be formed by gluing and/or thermal bonding.

If the core wrap is formed by two substrates 16, 16′, four seals may be used to enclose the absorbent material 60 within the core wrap. For example, a first substrate 16 may be placed on one side of the core (the top side as represented in the Figures) and extend around the core's longitudinal edges to at least partially wrap the opposed bottom side of the core. The second substrate 16′ may be present between the wrapped flaps of the first substrate 16 and the absorbent material 60. The flaps of the first substrate 16 may be glued to the second substrate 16′ to provide a strong seal. This so called C-wrap construction may provide benefits such as improved resistance to bursting in a wet loaded state compared to a sandwich seal. The front side and rear side of the core wrap may then also be sealed by gluing the first substrate and second substrate to another to provide complete encapsulation of the absorbent material across the whole of the periphery of the core. For the front side and rear side of the core, the first and second substrates may extend and may be joined together in a substantially planar direction, forming for these edges a so-called sandwich construction. In the so-called sandwich construction, the first and second substrates may also extend outwardly on all sides of the core and be sealed flat, or substantially flat, along the whole or parts of the periphery of the core typically by gluing and/or heat/pressure bonding. In an example, neither the first nor the second substrates need to be shaped, so that they may be rectangularly cut for ease of production but other shapes are within the scope of the present disclosure.

The core wrap may also be formed by a single substrate which may enclose as in a parcel wrap the absorbent material and be sealed along the front side and rear side of the core and one longitudinal seal.

### SAP Deposition Area

The absorbent material deposition area 8 may be defined by the periphery of the layer formed by the absorbent material 60 within the core wrap, as seen from the top side of the absorbent core. The absorbent material deposition area 8 may have various shapes, in particular, a so-called “dog bone” or “hour-glass” shape, which shows a tapering along its width towards the middle or “crotch” region of the core. In this way, the absorbent material deposition area 8 may have a relatively narrow width in an area of the core intended to be placed in the crotch region of the absorbent article, as illustrated in FIG. 1. This may provide better wearing comfort. The absorbent material deposition area 8 may also be generally rectangular, for example as shown in FIGS. 4-6, but other deposition areas, such as a rectangular, “T,” “Y,” “sand-hour,” or “dog-bone” shapes are also within the scope of the present disclosure. The absorbent material may be deposited using any suitable techniques, which may allow relatively precise deposition of SAP at relatively high speed.

### Channels

The absorbent material deposition area 8 may comprise at least one channel 26, which is at least partially oriented in the longitudinal direction of the article 80 (i.e., has a longitudinal vector component). Other channels may be at least partially oriented in the lateral direction (i.e., has a lateral vector component) or in any other direction. In the following, the plural form “channels” will be used to mean “at least one channel”. The channels may have a length L′ projected on the longitudinal axis 80 of the article that is at least 10% of the length L of the article. The channels may be formed in various ways. For example, the channels may be formed by zones within the absorbent material deposition area 8 which may be substantially free of, or free of, absorbent material, in particular SAP. In addition or alternatively, the channel(s) may also be formed by continuously or discontinuously bonding the top side of the core wrap to the bottom side of the core wrap through the absorbent material deposition area 8. The channels may be continuous but it is also envisioned that the channels may be intermittent. The acquisition-distribution system or layer 50, or another layer of the article, may also comprise channels, which may or not correspond to the channels of the absorbent core.

In some instances, the channels may be present at least at the same longitudinal level as the crotch point C or the lateral axis 60 in the absorbent article, as represented in FIG. 1 with the two longitudinally extending channels 26, 26′. The channels may also extend from the crotch region 7 or may be present in the front waist region 5 and/or in the rear waist region 6 of the article.

The absorbent core 28 may also comprise more than two channels, for example, at least 3, at least 4, at least 5, or at least 6 or more. Shorter channels may also be present, for example in the rear waist region 6 or the front waist region 5 of the core as represented by the pair of channels 27, 27′ in FIG. 1 towards the front of the article. The channels may comprise one or more pairs of channels symmetrically arranged, or otherwise arranged relative to the longitudinal axis 80.

The channels may be particularly useful in the absorbent core when the absorbent material deposition area is rectangular, as the channels may improve the flexibility of the core to an extent that there is less advantage in using a non-rectangular (shaped) core. Of course channels may also be present in a layer of SAP having a shaped deposition area.

The channels may be completely oriented longitudinally and parallel to the longitudinal axis or completely oriented transversely and parallel to the lateral axis, but also may have at least portions that are curved.

In order to reduce the risk of fluid leakages, the longitudinal main channels may not extend up to any of the edges of the absorbent material deposition area 8, and may therefore be fully encompassed within the absorbent material deposition area 8 of the core. The smallest distance between a channel and the closest edge of the absorbent material deposition area 8 may be at least 5 mm.

The channels may have a width We along at least part of their length which is at least 2 mm, at least 3 mm, at least 4 mm, up to for example 20 mm, 16 mm, or 12 mm, for example. The width of the channel(s) may be constant through substantially the whole length of the channel or may vary along its length. When the channels are formed by absorbent material-free zone within the absorbent material deposition area 8, the width of the channels is considered to be the width of the material free zone, disregarding the possible presence of the core wrap within the channels. If the channels are not formed by absorbent material free zones, for example mainly though bonding of the core wrap through the absorbent material zone, the width of the channels is the width of this bonding.

At least some or all of the channels may be permanent channels, meaning their integrity is at least partially maintained both in the dry state and in the wet state. Permanent channels may be obtained by provision of one or more adhesive materials, for example, the fibrous layer of adhesive material or construction glue that helps adhere a substrate with an absorbent material within the walls of the channel. Permanent channels may also be formed by bonding the upper side and lower side of the core wrap (e.g., the first substrate 16 and the second substrate 16′) and/or the topsheet 24 to the backsheet 25 together through the channels. Typically, an adhesive may be used to bond both sides of the core wrap or the topsheet and the backsheet through the channels, but it is possible to bond via other known processes, such as pressure bonding, ultrasonic bonding, heat bonding, or combination thereof. The core wrap or the topsheet 24 and the backsheet 25 may be continuously bonded or intermittently bonded along the channels. The channels may advantageously remain or become visible at least through the topsheet and/or backsheet when the absorbent article is fully loaded with a fluid. This may be obtained by making the channels substantially free of SAP, so they will not swell, and sufficiently large so that they will not close when wet. Furthermore, bonding the core wrap to itself or the topsheet to the backsheet through the channels may be advantageous.

### Barrier Leg Cuffs

The absorbent article may comprise a pair of barrier leg cuffs 34. Each barrier leg cuff may be formed by a piece of material which is bonded to the article so it may extend upwards from a wearer-facing surface of the absorbent article and provide improved containment of fluids and other body exudates approximately at the junction of the torso and legs of the wearer. The barrier leg cuffs are delimited by a proximal edge 64 joined directly or indirectly to the topsheet 24 and/or the backsheet 25 and a free terminal edge 66, which is intended to contact and form a seal with the wearer's skin. The barrier leg cuffs 34 extend at least partially between the front waist edge 10 and the rear waist edge 12 of the absorbent article on opposite sides of the longitudinal axis 80 and are at least present at the level of the crotch point (C) or crotch region. The barrier leg cuffs may be joined at the proximal edge 64 with the chassis of the article by a bond 65 which may be made by gluing, fusion bonding, or a combination of other suitable bonding processes. The bond 65 at the proximal edge 64 may be continuous or intermittent. The bond 65 closest to the raised section of the leg cuffs delimits the proximal edge 64 of the standing up section of the leg cuffs.

The barrier leg cuffs may be integral with the topsheet 24 or the backsheet 25 or may be a separate material joined to the article's chassis. Each barrier leg cuff 34 may comprise one, two or more elastic strings 35 close to the free terminal edge 66 to provide a better seal.

In addition to the barrier leg cuffs 34, the article may comprise gasketing cuffs 32, which are joined to the chassis of the absorbent article, in particular to the topsheet 24 and/or the backsheet 25 and are placed externally relative to the barrier leg cuffs. The gasketing cuffs 32 may provide a better seal around the thighs of the wearer. Each gasketing leg cuff may comprise one or more elastic strings or elastic elements 33 in the chassis of the absorbent article between the topsheet 24 and backsheet 25 in the area of the leg openings. All, or a portion of, the barrier leg cuffs and/or gasketing cuffs may be treated with a lotion or another skin care composition.

### Acquisition-Distribution System

The absorbent articles of the present disclosure may comprise an acquisition-distribution layer or system 50 (“ADS”). One function of the ADS is to quickly acquire one or more of the fluids and distribute them to the absorbent core in an efficient manner. The ADS may comprise one, two or more layers, which may form a unitary layer or may remain as discrete layers which may be attached to each other. In an example, the ADS may comprise two layers: a distribution layer 54 and an acquisition layer 52 disposed between the absorbent core and the topsheet, but the present disclosure is not so limited.

The ADS may comprise SAP as this may slow the acquisition and distribution of the fluids. Suitable ADS are described in WO 2000/59430 (Daley), WO 95/10996 (Richards), U.S. Pat. No. 5,700,254 (McDowall), and WO 02/067809 (Graef), for example.

In one example, the ADS may not be provided, or only one layer of the ADS may be provided, such as the distribution layer only or the acquisition layer only. When one of the three-dimensional, liquid permeable substrates of the present disclosure is used as a portion of, or all of, a topsheet, or positioned on a topsheet, dryness performance of the liquid permeable substrates may be improved if only one or no layers of the ADS are present. This is owing to the fact that fluids (e.g., urine) are easily able to wick through the liquid permeable substrates directly into the absorbent core 28 and/or into a one layer ADS.

### Distribution Layer

The distribution layer of the ADS may comprise at least 50% by weight of cross-linked cellulose fibers. The cross-linked cellulosic fibers may be crimped, twisted, or curled, or a combination thereof including crimped, twisted, and curled. This type of material is disclosed in U.S. Pat. Publ. No. 2008/0312622 A1 (Hundorf). The cross-linked cellulosic fibers provide higher resilience and therefore higher resistance to the first absorbent layer against the compression in the product packaging or in use conditions, e.g., under wearer weight. This may provide the core with a higher void volume, permeability, and liquid absorption, and hence reduced leakage and improved dryness.

The distribution layer comprising the cross-linked cellulose fibers of the present disclosure may comprise other fibers, but this layer may advantageously comprise at least 50%, or 60%, or 70%, or 80%, or 90%, or even up to 100%, by weight of the layer, of cross-linked cellulose fibers (including the cross-linking agents). Examples of such mixed layer of cross-linked cellulose fibers may comprise about 70% by weight of chemically cross-linked cellulose fibers, about 10% by weight polyester (PET) fibers, and about 20% by weight untreated pulp fibers. In another example, the layer of cross-linked cellulose fibers may comprise about 70% by weight chemically cross-linked cellulose fibers, about 20% by weight lyocell fibers, and about 10% by weight PET fibers. In still another example, the layer may comprise about 68% by weight chemically cross-linked cellulose fibers, about 16% by weight untreated pulp fibers, and about 16% by weight PET fibers. In yet another example, the layer of cross-linked cellulose fibers may comprise from about 90 to about 100% by weight chemically cross-linked cellulose fibers.

### Acquisition Layer

The ADS 50 may comprise an acquisition layer 52. The acquisition layer may be disposed between the distribution layer 54 and the topsheet 24. The acquisition layer 52 may be or may comprise a nonwoven material, such as a hydrophilic SMS or SMMS material, comprising a spunbonded, a melt-blown and a further spunbonded layer or alternatively a carded staple fiber chemical-bonded nonwoven. The nonwoven material may be latex bonded.

A further acquisition layer may be used in addition to a first acquisition layer described above. For example, a tissue layer may be placed between the first acquisition layer and the distribution layer. The tissue may have enhanced capillarity distribution properties compared to the acquisition layer described above.

Relationship between the Layers

Typically, adjacent layers and components may be joined together using conventional bonding methods, such as adhesive coating via slot coating or spraying on the whole or part of the surface of the layer, thermo-bonding, pressure bonding, or combinations thereof. This bonding is not represented in the Figures (except for the bonding between the raised elements of the leg cuffs 65 with the topsheet 24) for clarity and readability, but bonding between the layers of the article should be considered to be present unless specifically excluded. Adhesives may be used to improve the adhesion of the different layers between the backsheet 25 and the core wrap. The glue may be any suitable hot melt glue known in the art.

### Fastening System

The absorbent article may include a fastening system. The fastening system may be used to provide lateral tensions about the circumference of the absorbent article to hold the absorbent article on the wearer as is typical for taped diapers. The fastening system may comprise a male fastener that that includes hooks, and a female fastening material that comprises loops for engaging the hooks of the male fastener. The outer cover non-woven of the backsheet, as detailed above, may be utilized as an integral female fastening material. In other embodiments, the female fastening material may be a separate landing zone attached to the backsheet, the landing zone being constructed of the bonded outer cover non-woven material detailed above. Examples of male and female fasteners of fastening systems that are useful on the absorbent articles detailed herein are described in U.S. Patent Publication No. 2013/0123738, filed on Nov. 10, 2011.

### Female Fastening Material:

The following description relates to the female fastening material of the fastening system, which may be integral to the outer cover non-woven of the backsheet. Any of the following aspects of the female fastening material may be combined with any of the aspects of the bonding pattern of the outer cover non-woven material that are detailed above.

Suitable nonwoven web materials useful in the present invention include, but are not limited to spunbond, meltblown, spunmelt, solvent-spun, electrospun, carded, film fibrillated, melt-film fibrillated, air-laid, dry-laid, wet-laid staple fibers, and other and other nonwoven web materials formed in part or in whole of polymer fibers, as known in the art. A suitable nonwoven web material may also be an SMS material, comprising a spunbonded, a melt-blown and a further spunbonded stratum or layer or any other combination of spunbonded and melt-blown layers, such as a SMMS or SSMMS etc. Examples include one or more layers of fibers with diameters below 1 micron (nanofibers and nanofiber layers); examples of these rise in combinations of SMS, SMNS, SSMNS or SMNMS nonwoven webs (where “N” designates a nanofiber layer). In some examples, permanently hydrophilic non-wovens, and in particular, nonwovens with durably hydrophilic coatings may be desirable. Typically, the suitable non-woven is air permeable. Typically the suitable nonwoven is water or liquid permeable, but may also be water impermeable by reason of fiber size and density, and hydrophobicity of the fibers. Water or liquid permeability may be enhanced by treatments to render the fibers hydrophilic, as discussed below.

The nonwoven web may be formed predominately of polymeric fibers. In some examples, suitable non-woven fiber materials may include, but are not limited to polymeric materials such as polyolefins, polyesters, polyamide, or specifically polypropylene (PP), polyethylene (PE), poly-lactic acid (PLA), polyethylene terephthalate (PET) and/or blends thereof. Nonwoven fibers may be formed of, or may include as additives or modifiers, components such as aliphatic polyesters, thermoplastic polysaccharides, or other biopolymers (bio-based or renewable polymers).

The individual fibers may be monocomponent or multicomponent. The multicomponent fibers may be bicomponent, such as in a core-and-sheath or side-by-side arrangement. Often, the individual components comprise aliphatic polyolefins such as polypropylene or polyethylene, or their copolymers, aliphatic polyesters, thermoplastic polysaccharides or other biopolymers.

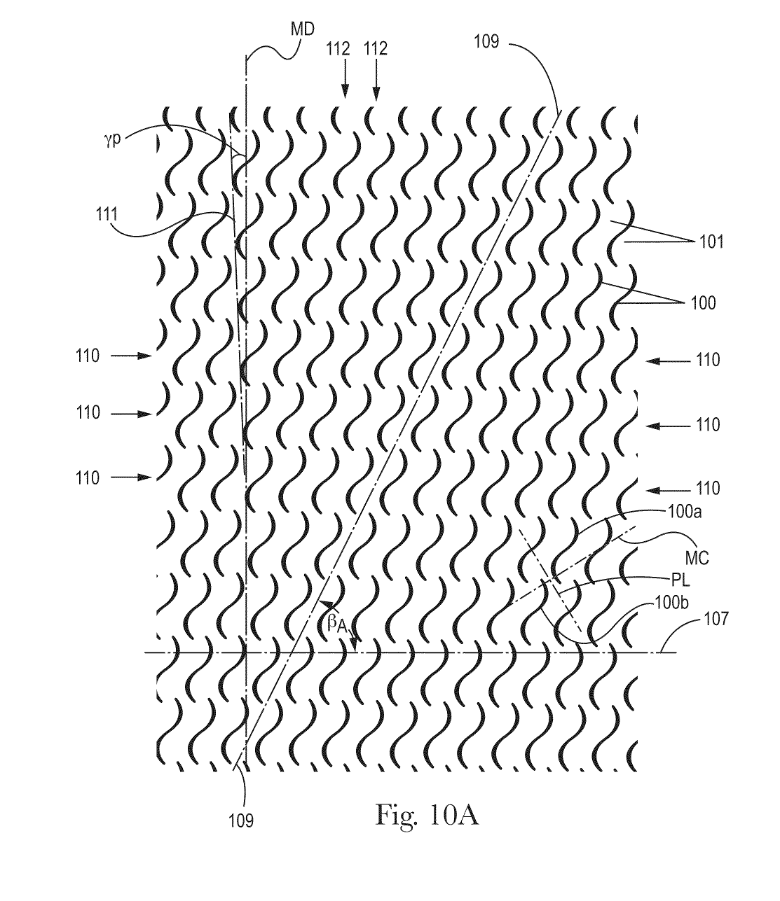

FIG. 11B is a further enlarged cross-sectional view of a fiber 314 from the female fastening material 310 of FIG. 11A. The fiber 314 has an overall fiber cross-sectional dimension 315. Since the shape of the fiber 314 is round, the overall cross-sectional shape of the fiber 314 is circular, and the overall fiber cross-sectional dimension 315 is the diameter of the circular overall cross-sectional shape of the fiber 314. For fibers with non-circular cross-sections, the overall fiber cross-sectional dimension is the largest distance measured linearly across the cross-section of the fiber. The overall fiber cross-sectional dimension of a fiber can be determined by making measurements on an enlarged image of the fiber, taken with equipment, at high magnification. For example, a Scanning Electron Microscope can be used.

The female fastening material 310 can include fibers of various sizes and shapes. For example, some or all of fibers in the female fastening material 310 can have an overall cross-sectional dimension of 5-150 micrometers, or any integer value for micrometers between 5 and 150 micrometers, or any range formed by any of these values. As examples, the overall thickness can be less than 25 micrometers, less than 20 micrometers, or less than 15 micrometers or any range formed by any of these values. Also as an example, some or all of the fibers in the female fastening material 310 can have an overall cross-sectional shape that is circular, oval, squarish, rectangular, triangular, star-shaped, multi-lobal, or any other shape known in the art, or combinations of any of these. As an example, a fibrous material used as a female fastening material in a hook and loop fastening system, can be a nonwoven material made from bicomponent fibers, having an overall cross-sectional dimension of 17 micrometers and an overall cross-sectional shape that is round.

### Male Fastener:

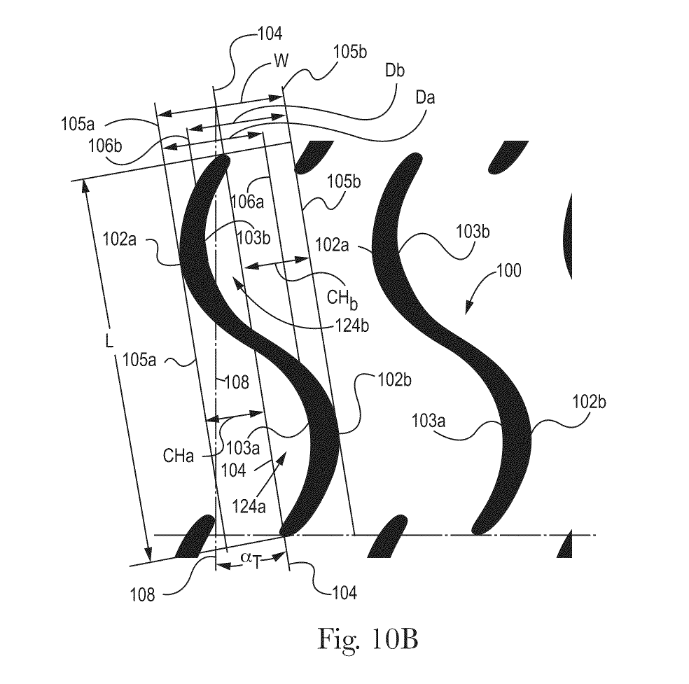

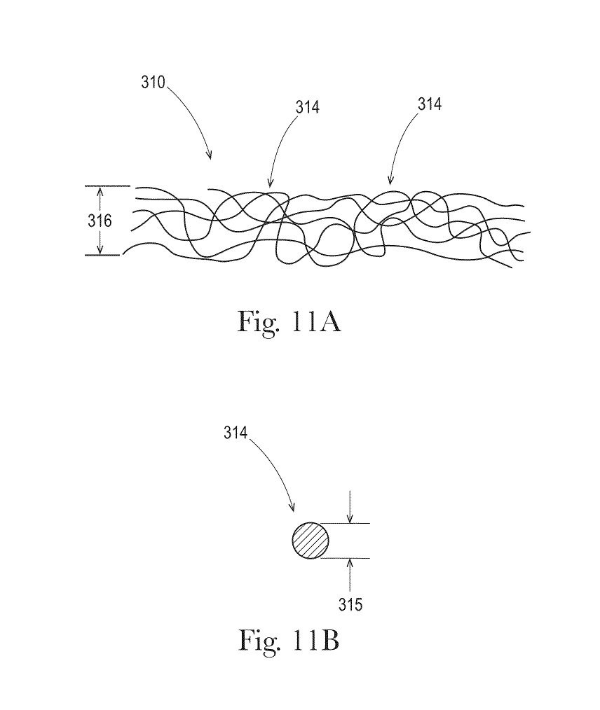

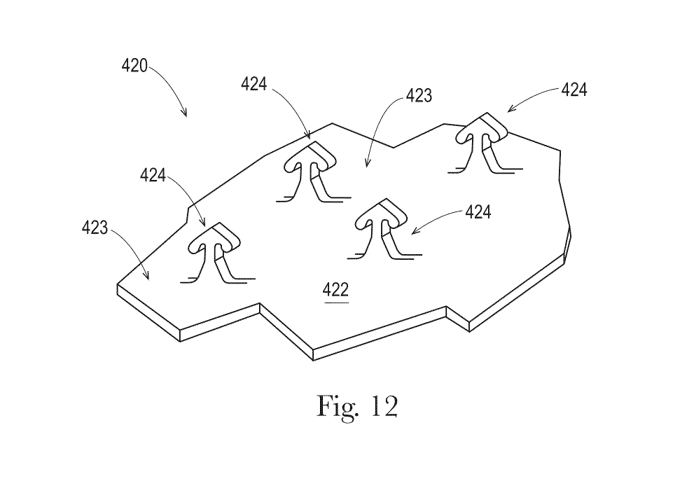

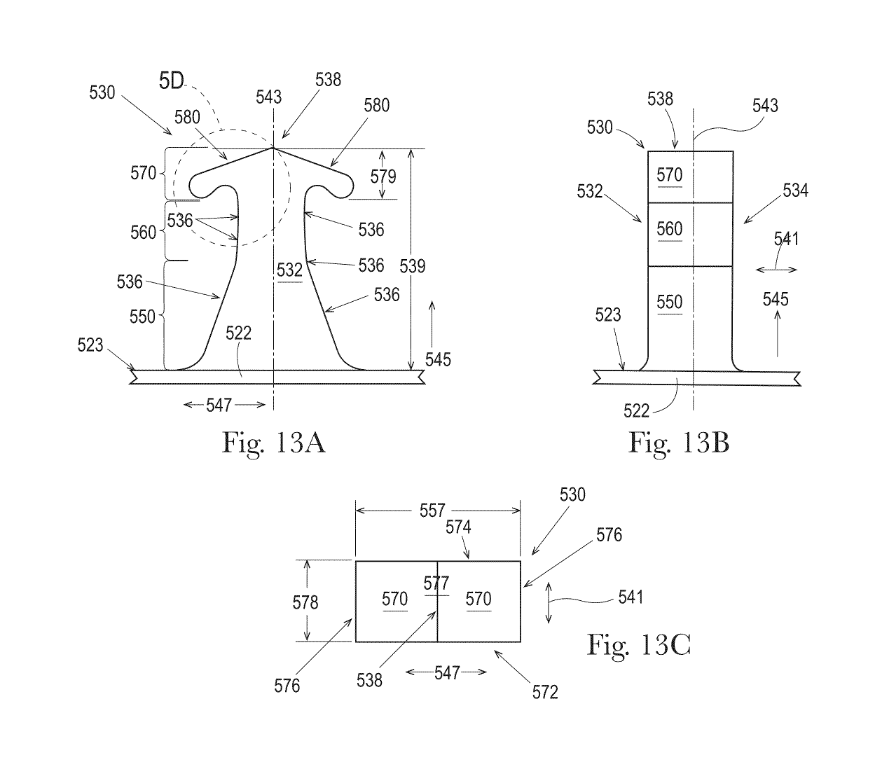

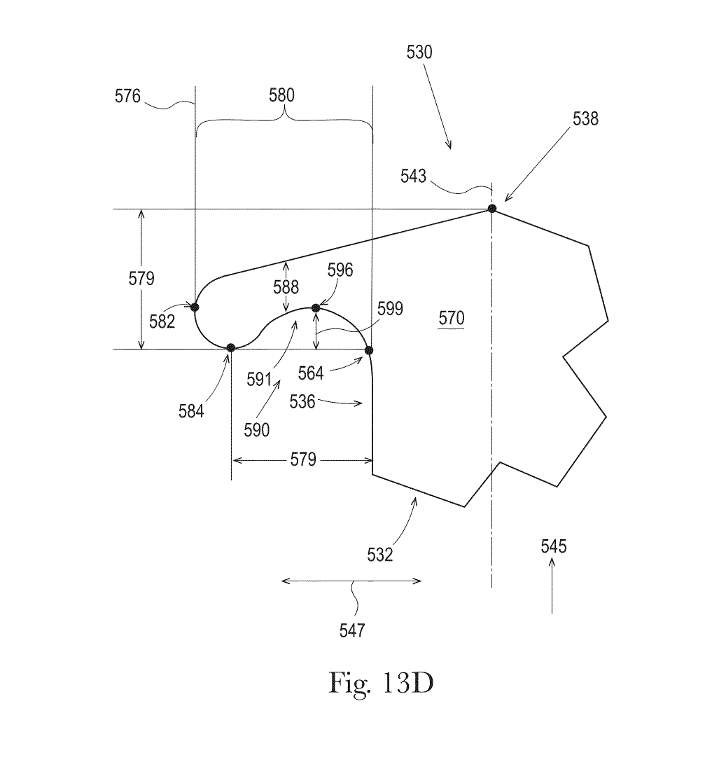

FIG. 12 is an enlarged isometric view of a portion of a male fastening material 420. In FIG. 12, the portion is shown as broken away from a larger piece of material. The male fastening material 420 includes a plurality of bidirectional micro-sized hooks 424 disposed on a substrate 422 that has an overall planar shape. Each of the hooks 424 can be configured in the same way as the hook 530 of FIGS. 13A-13D, including any alternative embodiments. Alternatively the male fastening material 420 can also include one or more hooks configured in other ways. In the embodiment of FIG. 12, the hooks 424 and the substrate 422 are made from the same material, and the hooks 424 are a unified part of the substrate 422. The male fastening material 420 can be made from a wide variety of shapable and/or formable materials, including any of the natural or synthetic materials recited herein and/or any other suitable material suitable known in the art, in any workable combination, along with any additives or processing aids known in the art. As a particular example, the male fastening material 420 can be made from various renewable materials, including bioplastics derived from renewable biomass sources such as sugars, starches, cellulose, biopolymers, etc. In various alternate embodiments, the hooks and the substrate may be formed separately, or made from different materials.

The hooks 424 can be distributed across the male fastening material 420 in various patterns and hook densities. For example, the hooks 424 can be arranged in rows and/or columns, or any other arrangement of hooks known in the art. In various embodiments, the male fastening material can have a hook density of 10-1,000 hooks per square centimeter, or any integer number of hooks between 10 and 1,000, or any range formed by any of these values.

The male fastening material 420 and the hooks 424 thereon can be made by any suitable process known in the art. For example, the male fastening material can be made by casting, molding, profile extrusion, or microreplication.

FIG. 13A is an enlarged view of a front 532 of a bidirectional micro-sized hook 530 disposed on a top surface 523 of a portion of a substrate 522 of a male fastening material. In FIG. 13A, the portion of the substrate 522 is shown as broken away from a larger piece of material. The hook 530 is bidirectional since the cap 570 has two arms 580, extending from opposite sides 536 of the hook 530. The sides 536 of the hook are the outside portions of the hook 530, between a front 532 of the hook 530 and a back 534 of the hook 530. While the hook 530 is a bidirectional hook, it is contemplated that any of the structures, features, sizes, or dimensions of the hook 530 can be similarly applied to a unidirectional hook (one arm configured to hook in one direction) or to a multi-directional hook (more than two arms, with each arm configured to hook in a different direction).

FIG. 13A shows a cross-section of the hook 530 that has a width direction 547. This width direction 547 is parallel to the cross direction of the hook web. FIG. 13B shows a hook 530 that has a thickness direction 541, which is parallel to the substrate 522 and perpendicular to the width direction 547. This thickness direction 541 is also parallel to the longitudinal axis direction 80 of the absorbent article on FIG. 4.

In the embodiment of FIG. 13A, both of the arms 580 of the hook 530 are configured in the same way. However, it is also contemplated that any of the structures, features, sizes, or dimensions of an arm 580 of the hook 530 can be applied to one arm of a bidirectional hook, while the other arm can be configured in a different way. Further, it is contemplated that any of the structures, features, sizes, or dimensions of an arm 580 of the hook 530 can be applied to two or more arms of a multi-directional hook, while one or more other arms on that hook can be configured in one or more different ways.

The hook 530 includes a base 550, a stem 560, and a cap 570. The hook 530 also has sides 536. The hook 530 projects out from the substrate 522 in an upward direction 545, which is perpendicular to the substrate 522. The hook 530 also has a width direction 547. The width direction 547 is parallel to the overall planar shape of the substrate 522 and parallel to the largest linear dimension measured across the cap 570. In the embodiment of FIG. 13A, the width direction 547 is also parallel to the front 532 of the hook 530 and perpendicular to the sides 536. The hook 530 also has a thickness direction 541, which is parallel to the substrate 522 and perpendicular to the width direction 547. The thickness direction 541 is perpendicular to the page in FIG. 13A, so the thickness direction 541 is shown in FIGS. 13B and 13C. The thickness direction 541 is also parallel to the machine direction of the hook web and the longitudinal direction of the article 80 on FIG. 4.

The base 550 is attached to the substrate 522, the stem 560 is attached to the base 550, and the cap 570 is attached to the stem 560. The cap 570 has an overall cap height 579, which is measured as described in connection with FIG. 13D. The hook 530 also has an overall hook height 539 measured linearly in the upward direction 545 from the top surface 523 of the substrate 522 to a highest point on the outer surface of the hook 530 (farthest away from the top surface 523 of the substrate 522). In the embodiment of FIG. 13A, a peak 538 of the cap 570 is the highest point on the outer surface of the hook 530. The hook 530 also has a central axis 543, which passes through the center of the hook. In the embodiment of FIG. 13A, the central axis 543 is aligned with the upward direction 545, however, in various embodiments, the central axis 543 may not be perpendicular with respect to the substrate 522; that is the hook may be tipped in the width direction 547 and/or the thickness direction 541. Also, in various embodiments the cap 570 may not have a peak, but may have a rounded top, or a flat top, or a recessed top, or any other shape known in the art or combinations of any of these.

The front edge 572, the back edge 574, and the side edges 576 together, when viewed from above the peak 538 of the hook 530, define a vertical engagement effective area 577. The vertical effective engagement area 577 has a width-to-thickness aspect ratio, which is defined as the widest overall width of the vertical engagement effective area 577 divided by the thickest overall thickness of the vertical engagement effective area 577. The width-to-thickness aspect ratio can be 1-2, or any value in increments of 0.01 between 1 and 2, or any range formed by any of these values. As examples, the width-to-thickness aspect ratio can be 1.2, 1.3, 1.6, 1.8, or 1.9, or any range formed by any of these values. The size of the vertical effective engagement area 577 can be 40,000-120,000 square micrometers, or any value in increments of 5,000 square micrometers between 40,000 and 120,000 square micrometers, or any range formed by any of these values. As examples, the vertical effective engagement area 577 can be 40,000, 50,000, 60,000, 90,000, 100,000, or 120,000 square micrometers, or any range formed by any of these values.

The recessed portion 591 has an overall recessed width 597 measured linearly in the width direction 547 from the lowest point 584 on the arm 580 (as described above) to the side point 564. When the hook 530 is used on a male fastening material of a fastening system, and the fastening system uses a fibrous material as the female fastening material, the overall recessed width 597 can be sized to the overall fiber cross-sectional dimension of the fibrous material, such as the overall fiber cross-sectional dimension 315 of the fiber 314 of the female fastening material 310. The overall recessed width 597 can be 200-500% of the overall fiber cross-sectional dimension, or any integer value of percentage between 200% and 500% of the overall fiber cross-sectional dimension, or any range formed by any of these values. As examples, the overall recessed width 597 can be 200%, 300%, 400%, or 500% of the overall fiber cross-sectional dimension, or any range formed by any of these values. While not wishing to be bound by this theory, it is believed that, selecting these relative dimensions for the overall recessed width 597, as described above, allow two to five fibers to fit in the recessed portion 591, which increases the likelihood that fibers can be retained by the hook 530.

When the hook 530 is used on a male fastening material of a fastening system, and the fastening system uses a fibrous material as the female fastening material, the deepest recessed depth 599 can be sized to the overall fiber cross-sectional dimension of the fibrous material, such as the overall fiber cross-sectional dimension 315 of the fiber 314 of the female fastening material 310. The deepest recessed depth 599 can be 40-200% of the overall fiber cross-sectional dimension, or any integer value of percentage between 40% and 200% of the overall fiber cross-sectional dimension, or any range formed by any of these values. As examples, the deepest recessed depth 599 can be 50%, 60%, 100%, or 150% of the overall fiber cross-sectional dimension, or any range formed by any of these values. While not wishing to be bound by this theory, it is believed that, selecting these relative dimensions for the deepest recessed depth 599, as described above, allows one or more fibers to fit in the recessed portion 591, and increases the likelihood that fibers will be retained by the hook 530.

The deepest recessed depth 599 can also be sized in relation to the overall cap height 579. The deepest recessed depth 599 can be 10-60% of the overall cap height 579, or any integer value of percentage between 10% and 50% of the overall cap height 579, or any range formed by any of these values. As examples, the deepest recessed depth 599 can be 10%, 20%, 50%, or 60% of the overall cap height 579, or any range formed by any of these values.

### Front and Rear Ears

The absorbent article may comprise front ears 46 and rear ears 40. The ears may be an integral part of the chassis, such as formed from the topsheet 24 and/or backsheet 26 as side panels. Alternatively, as represented on FIG. 1, the ears may be separate elements attached by gluing, heat embossing, and/or pressure bonding. The rear ears 40 may be stretchable to facilitate the attachment of the tabs 42 to the landing zone 44 and maintain the taped diapers in place around the wearer's waist. The rear ears 40 may also be elastic or extensible to provide a more comfortable and contouring fit by initially conformably fitting the absorbent article to the wearer and sustaining this fit throughout the time of wear well past when absorbent article has been loaded with fluids or other bodily exudates since the elasticized ears allow the sides of the absorbent article to expand and contract.

### Test Methods

### Vertical Pull with Constant Shear (VPT-S) Method

The intent of this method is to determine the performance of a fastener system at different combinations of applied force and constant shear. Material combinations must meet minimum peak load requirements to prevent unwanted in-use failures, such as pop-off.

### Equipment:

1. VPT-S lower and upper fixtures and motor (RDL-2195).
2. VPT-S weight set (RDL-2195).
3. MTS Tensile Tester with Test Works 4™ software.
4. Nylon string for weight application with the following specification: 2 mm-3 mm of stretch per 150 mm sample under 7 N of stress.
5. Cerro metal, 158° F. melting point.
6. Hot plate, capable of heating to 200° F.
7. Sample Prep tools: scissors, freeze spray, 3M 1524 Transfer Tape, 3M 9579 Double Coated Tape, tissue.

### Sample Preparation:

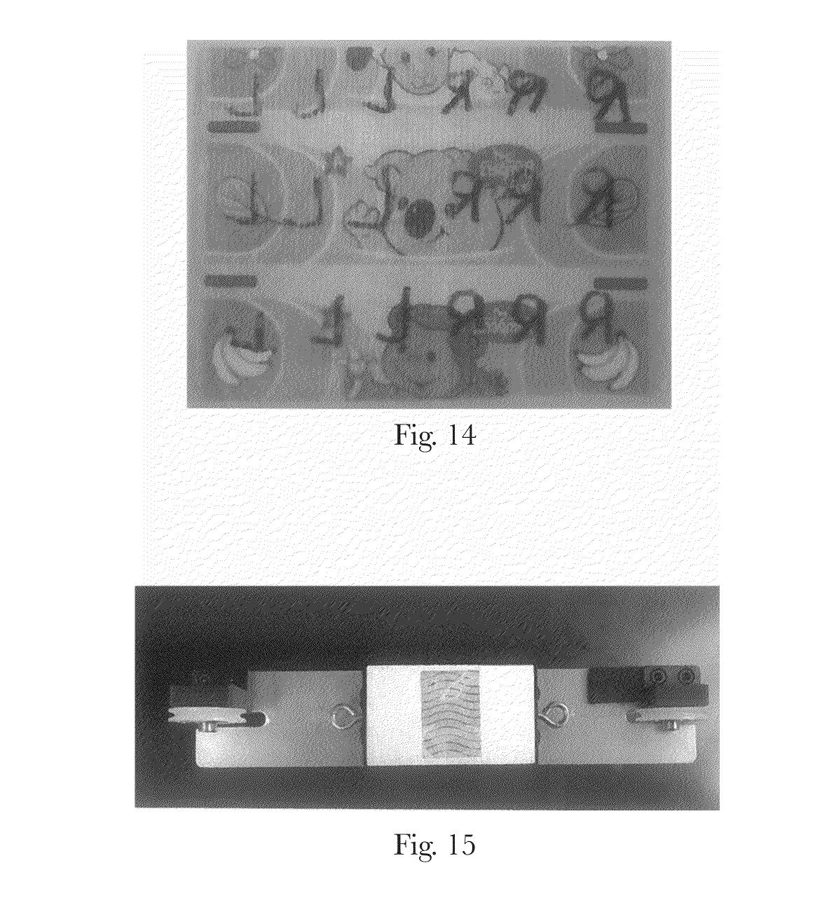

A) Sample Preparation for Female Fastener Material
1. Carefully remove the female fastener material from the finished product by cutting it off and remove all excess materials from the reverse side.
2. Turn the female fastener material over so that the loop side is facing down.
3. Write letter R backwards several times across the right side of the female fastening material. (see FIG. 14)
4. Write the letter L backwards several times across the left side of the female fastening material.
5. Attach a layer of 3M 1524 transfer adhesive to a strip of 3M Double Coated Tape 9579.
6. Remove the backing tape of the 3M 1524 and carefully without forming wrinkles, or bubbles, attach the female fastening material, loops upwards to the 3M 1524/9579 laminate. If bubbles or wrinkles are formed, do not use that portion of the sample for testing.
7. Cut female fastener material samples 50.8 mm (2″) MD by 25.4 mm (1″) CD from both left and right sides. If the MD female fastener material is smaller than 50.8 mm, cut to the largest possible dimension.
8. Carefully trim any free adhesive surface that remains on samples. Then, touch edges of sample with tissue to mask any adhesive residue.

B) Sample Preparation of the Male Fastener
1. Carefully remove the male fastener from the finished product using a freezing spray. Take care not to damage the hooks. If it is not possible to remove the hook patch without damage then carefully cut it off the product.
2. Attach a layer of 3M 1524 transfer adhesive to a strip of 3M Double Coated Tape 9579.
3. Remove the backing tape of the 3M 1524 and attach the hook material to the 3M 1524/9579 laminate, hooks facing up.
4. Cut male fastener samples 13 mm CD by 25.4 mm (1″) MD.
5. Carefully trim any free adhesive surface that remains on samples. Then, touch edges of sample with tissue to mask any adhesive residue.

### Run Preparation

1. Open in TestWorks™ the “VPT Constant Shear Method”.
2. Place female fastening material sample upside down with loops facing base plate. Attach hooks to upper plate (see Run Procedure step 5 for attachment instructions).
3. Zero the Load Cell meter. Then lower the crosshead manually until the application force equals −0.100N.
4. Zero the Displacement meter.
5. Using the MTS program, specify that the crosshead rise 50 mm from this position.
   a. Only perform the gauge length calibration once per set of hook/loop combination.
6. Zero the Displacement meter.

### Run Procedure

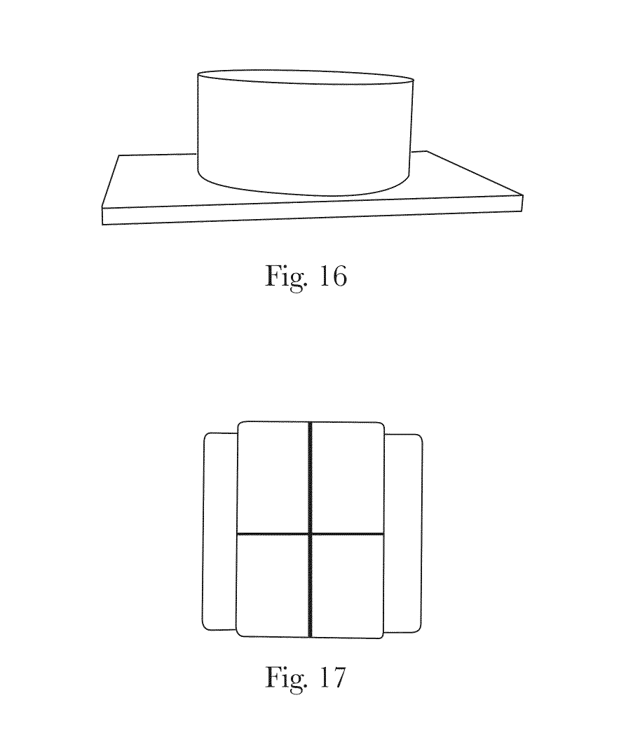

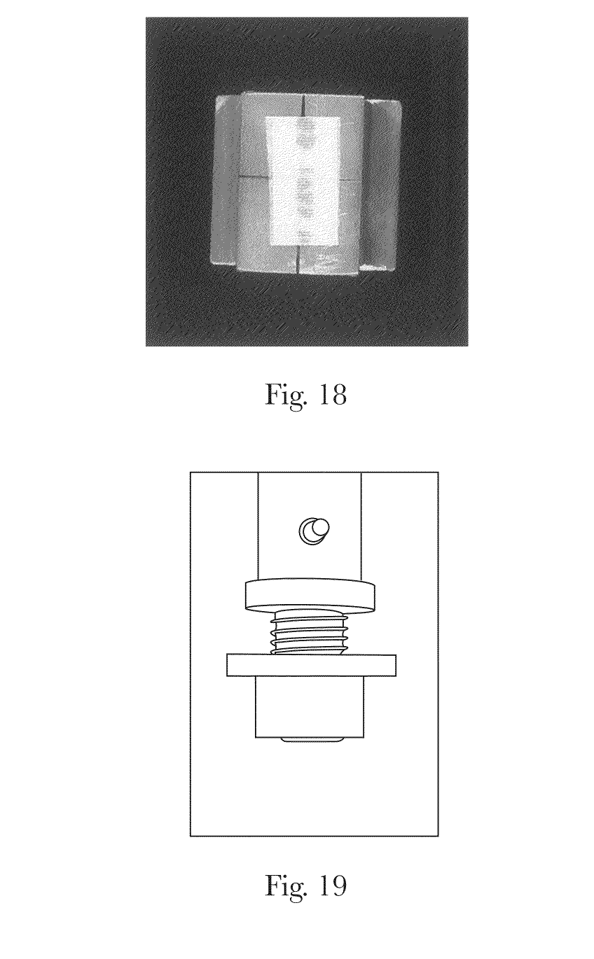

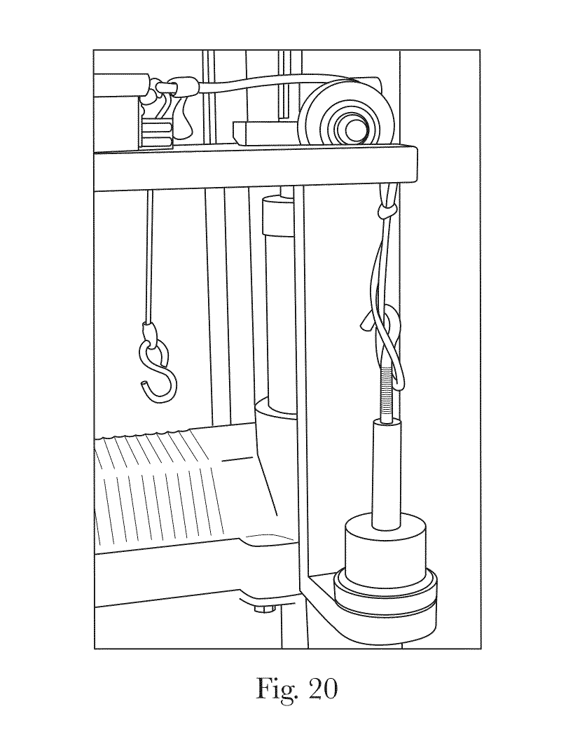

1. Remove the backing on the female fastener material sample. Orient with the CD going left to right on the lower plate and gently place the landing zone to the center of the plate as shown below (see FIG. 15).
2. Gently place 250 g weighted plate (at least 3 inches by 2 inches) on female fastener material (see FIG. 16). Without applying additional pressure, slide weight over entire sample surface to secure female fastener material to base plate.
3. The “R” labeled samples will be oriented upside-down, “L” labeled samples will oriented properly. Female fastener material samples are replaced every test, male fastener samples are replaced every other test.
4. Remove the backing on the male fastener sample. Attach the male fastener sample to the center of the upper plate so that the CD direction of the hooks will match that of the female fastener material. (see FIGS. 17 and 18)
5. Place the upper plate into upper section of the tensile tester. Orient upper plate with the Front Face (indicated by “F”) towards the operator. (see FIG. 19)
6. Position the sliding platform so that the female fastener material is centered underneath the male fastener sample.
7. Place the desired shear weight on the Actuator Arm, and set Arm to highest vertical position. (see FIG. 20)
8. Set the application force needed for the test in Test Works 4 program interface.
9. Make sure that the pulley cord is in the pulley's groove and that the cord loop will catch the weight.
10. Start the test. The upper plate should descend at 5 mm/s for the first 49 mm then changing to 0.5 mm/s until the application force is achieved. At this point a prompt will ask the operator to shear the sample.
11. Within 5 seconds of previous step, press the button to lower the Actuator Arm to its lowest vertical position. (see FIG. 21)
12. Within 5 seconds of the Actuator Arm reaching its lowest position, press <Enter> on the keyboard or click “OK” on the monitor.
13. The upper plate will move upward, the lower plate will move laterally as the weight falls, and the male fastener sample will disengage from the female fastener material.
14. Normally a hook can be used a maximum of 2 times before replacing. A new female fastener material sample is required each time.
15. Repeat Run Procedure steps 1-15 for as many samples as are needed.

### Data Collection/Analysis:

Recommended Test Space (9 Total Conditions, n=6 per Condition)
1) 0.100N application force with shear from 100 g, 400 g and 700 g weights
2) 0.550N application force with shear from 100 g, 400 g and 700 g weights
3) 1.00N application force with shear from 100 g, 400 g and 700 g weights

### Valid Data Criteria

1. If any delamination occurs (female fastener material from tape, tape from sled, hook from tape, tape from upper plate), make note in laboratory notebook, delete data point and re-run.
2. If sled moves laterally such that entire male fastener area is not on the female fastener material prior to starting the test, make note in laboratory notebook and keep data point. This is considered a zero data point—the data will be used, but this does not count towards the total number of specimens to be run (i.e. 8 total specimens including 2 zero data points would count as a 6 specimen dataset). This type of failure is called a “catastrophic failure” because the system is so weak it cannot even be tested to evaluate resistance to disengagement perpendicular to the surface. If all samples repeatedly catastrophically fail for any given test condition, record the result for that condition as zero and report “complete catastrophic failure” along with the remaining results. If any samples catastrophically fail for a given condition, report the number catastrophically failed and number attempted to be tested at that condition along with the remaining result.
3. Raw Data should display linear behavior at ˜0.05 mm to the peak

### Validated Data Outputs at Each Application Force/Shear Pairing

*Contour Plot (in JMP) recommended to analyze entire test space simultaneously for all output variables
1. Peak Load (N)—Maximum force recorded before disengagement occurs
2. Load (N) at 1.0 mm vertical displacement

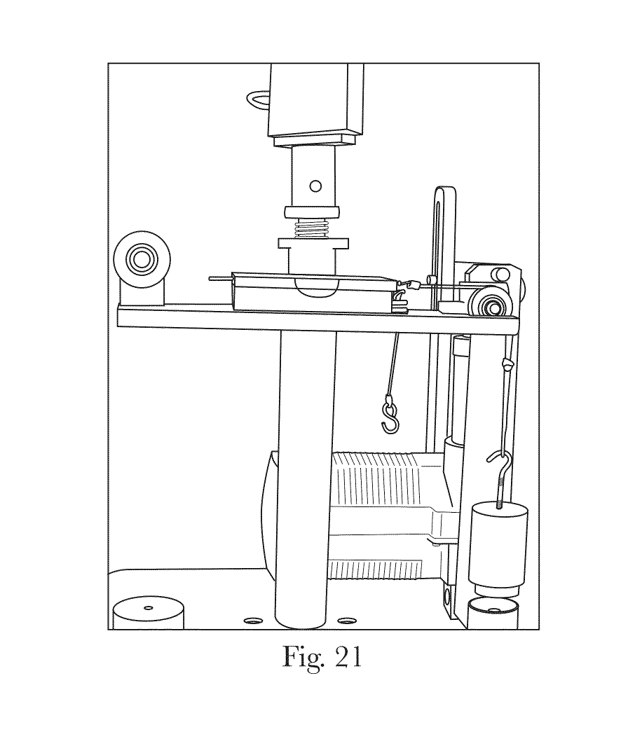

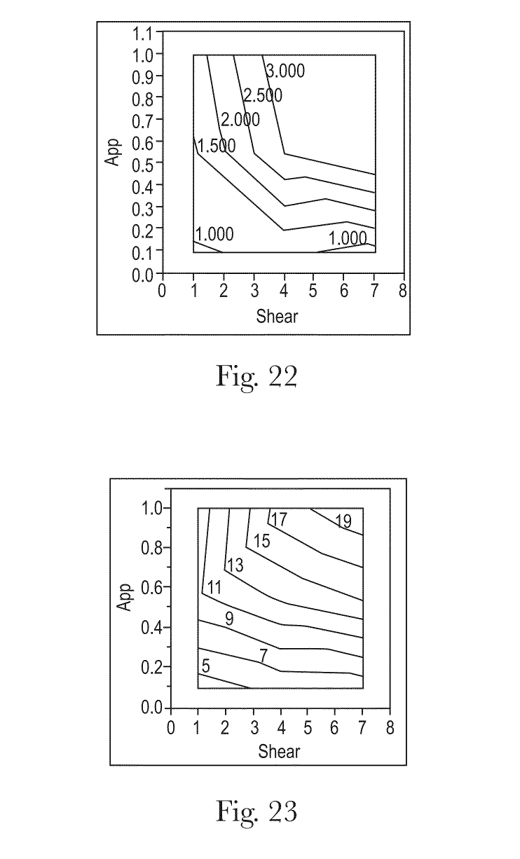

An example of a contour plot made utilizing JMP software is shown in FIG. 22. In particularly preferred embodiments, the system will provide suitable resistance to disengagement in a direction perpendicular to the surface of the fastening system according to the VPT-S test method. Further, it is particularly preferably to have a very low percentage of samples which catastrophically fail at any given test condition of the VPT-S test method. For example, less than 25%, less than 10% and more preferably 0% of samples fail at any given condition of the VPT-S test method. In particularly preferred systems, 0 out of 10 samples fail at the 0.1 N application force and 7 N shear force condition in the VPT-S test method.

### EXAMPLES

### Example 1

The fastener system of Example 1 is currently used on one embodiment of Pampers diapers. The male fastener is an Aplix 963 fastener, commercially available from Aplix, Inc. of Charlotte, N.C., USA under the trade designation 963. The female fastener material is EBL Bright, commercially available from the 3M. The EBL material is a separate landing zone that is attached to the backsheet (i.e., not integral to the backsheet). VPT-S testing as detailed above was performed on the fastener system of Example 1. The resulting contour plot is depicted as FIG. 23. The contour plot illustrates that the fastening system requires at least about 5 N to be disengaged in the direction perpendicular to the surface for the majority of the test condition space (e.g., range of application force and shear force applied), and in this particular case there were no points in which the sled moved laterally such that the entire male fastener area is not on the female fastener material prior to starting the test. This system provides sufficient performance to provide suitable performance in use.

### Example 2

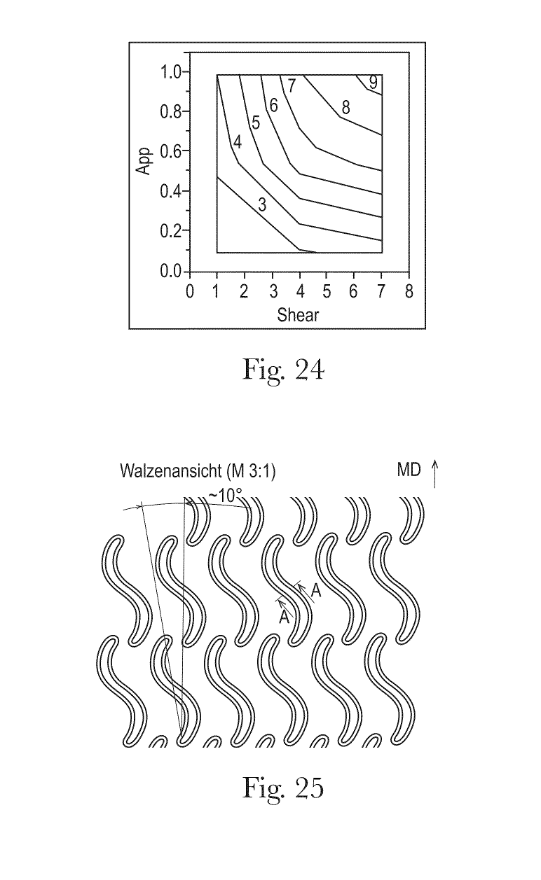

The fastener system of Example 2 is currently used on another embodiment of Pampers diapers. The male fastener is an Aplix 980 fastener, commercially available from Aplix, Inc. of Charlotte, N.C., USA under the trade designation 980. The female fastener material is NWLZ, which is currently used on commercially available Pampers and Luvs diapers. The NWLZ material is a separate landing zone that is attached to the backsheet (i.e., not integral to the backsheet). VPT-S testing as detailed above was performed on the fastener system of Example 2. The resulting contour plot is depicted as FIG. 24. The contour plot illustrates that the fastening system requires at least about 3 N to be disengaged in the direction perpendicular to the surface for the majority of the test condition space (e.g., range of application force and shear force applied) other than the space below about 4 N shear and below about 0.5 N application force, in which the system requires about 2 N. For this system, there were no points in which the sled moved laterally such that the entire male fastener area is not on the female fastener material prior to starting the test. This system provides sufficient performance to provide suitable performance in use.

### Example 3

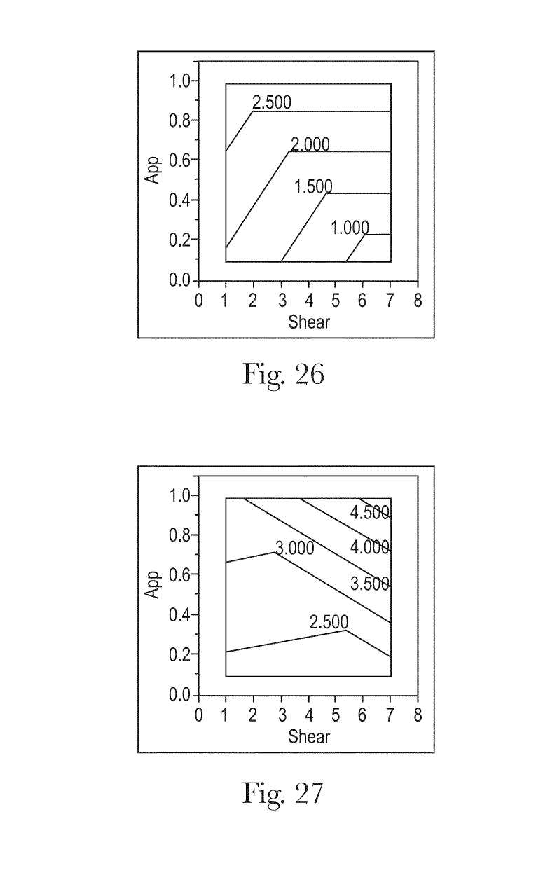

The fastener system of Example 3 includes a male fastener from 3M, known in the industry as XHK05636 and a female fastener integral to the backsheet. The female fastener material referred to as Soft Bond II (SB2) 13 gsm is a 13 gsm spunbond nonwoven made of polypropylene having average fiber diameter of 1.8 denier, manufactured by Pegas Nonwovens s.r.o. SB2 13 gsm was thermally bonded having a bond pattern, as shown in FIG. 25. VPT-S testing as detailed above was performed on the fastener system of Example 3. The resulting contour plot is depicted as FIG. 26. The contour plot illustrates that the fastening system requires at least about 1 N to be disengaged in the direction perpendicular to the surface for the majority of the test condition space (e.g., range of application force and shear force applied) other than the space above about 4 N shear and less than about 0.2 N application force, in which the system requires less than about 1 N. Many wearing conditions can provide a normal force above this level, so higher than typical failure rates (diaper becoming unfastened during wearing) may occur. Further, in this high shear, low application force portion of the space tested, all samples resulted in the sled moving laterally such that the entire male fastener area is not on the female fastener material prior to starting the test. This means the fastening system has a high risk of failing to keep the diaper closed during wearing for consumers who apply the diaper tightly and only lightly press the male fastener to engage it to the female fastener.

### Example 4

The fastener system of Example 4 includes a male fastener that is an Aplix 980 fastener and a female fastener integral to the backsheet. The female fastener material is Soft Bond II, at a basis weight of 13 gsm. The female fastener material referred to as Soft Bond II (SB2) 13 gsm is a 13 gsm spunbond nonwoven made of polypropylene having average fiber diameter of 1.8 denier, manufactured by Pegas Nonwovens s.r.o. SB2 13 gsm was thermally bonded having a bond pattern, as shown in FIG. 25. VPT-S testing as detailed above was performed on the fastener system of Example 4. The resulting contour plot is depicted as FIG. 27. The contour plot illustrates that the fastening system requires at least about 2.5 N to be disengaged in the direction perpendicular to the surface for the majority of the test condition space (e.g., range of application force and shear force applied) other than the space below about 0.2 N application force, in which the system requires about 2 N. For this system, there were no points in which the sled moved laterally such that the entire male fastener area is not on the female fastener material prior to starting the test. This system provides sufficient performance to provide suitable performance in use.

### Example 5

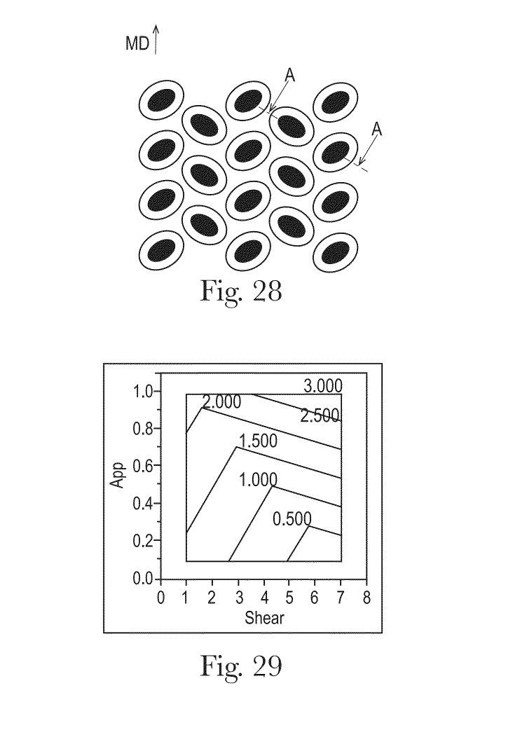

The fastener system of Example 5 includes a male fastener that is a XHK05636 fastener and a female fastener integral to the backsheet. The female fastener material is a 15 gsm spunbond nonwoven made of polypropylene having average fiber diameter of 1.8 denier, manufactured by Guelsan Sentetik. The spunbond 15 gsm was thermally bonded having a bond pattern, as shown in FIG. 28. VPT-S testing as detailed above was performed on the fastener system of Example 5. The resulting contour plot is depicted as FIG. 29. The contour plot illustrates that the fastening system requires at least about 0.5 N to be disengaged in the direction perpendicular to the surface for the majority of the test condition space (e.g., range of application force and shear force applied) other than the space above about 5 N shear and less than about 0.2 N application force, in which the system requires between about 0 and about 0.5 N. Many wearing conditions can provide a normal force above this level, so higher than typical failure rates (diaper becoming unfastened during wearing) may occur. Further, in this high shear, low application force portion of the space tested, all samples resulted in the sled moving laterally such that the entire male fastener area is not on the female fastener material prior to starting the test. This means the fastening system has a high risk of failing to keep the diaper closed during wearing for consumers who apply the diaper tightly and only lightly press the male fastener to engage it to the female fastener.

### Example 6

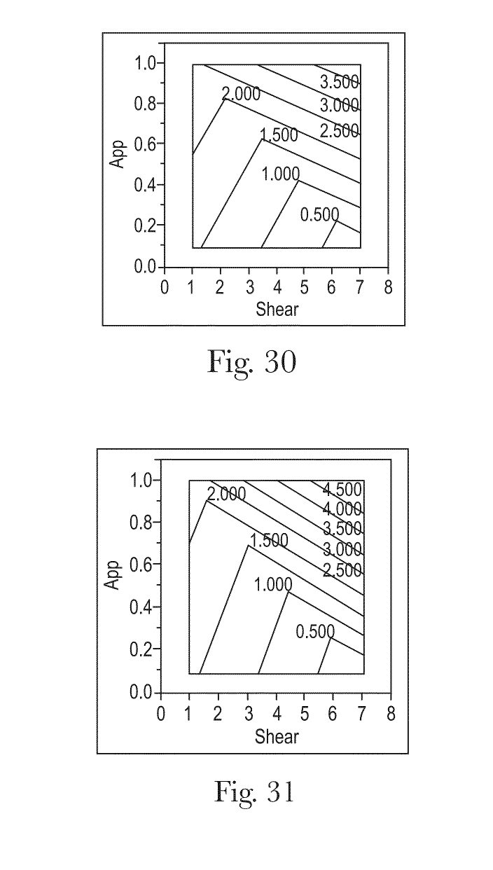

The fastener system of Example 6 includes a male fastener that is an Aplix 980 fastener and a female fastener integral to the backsheet. The female fastener material is a 15 gsm spunbond nonwoven made of polypropylene having average fiber diameter of 1.8 denier, manufactured by Guelsan Sentetik. The spunbond 15 gsm was thermally bonded having a bond pattern, as shown in FIG. 28. VPT-S testing as detailed above was performed on the fastener system of Example 6. The resulting contour plot is depicted as FIG. 30. The contour plot illustrates that the fastening system requires at least about 0.5 N to be disengaged in the direction perpendicular to the surface for the majority of the test condition space (e.g., range of application force and shear force applied) other than the space above about 5 N shear and less than about 0.2 N application force, in which the system requires between about 0 and about 0.5 N. Many wearing conditions can provide a normal force above this level, so higher than typical failure rates (diaper becoming unfastened during wearing) may occur. Further, in this high shear, low application force portion of the space tested, all samples resulted in the sled moving laterally such that the entire male fastener area is not on the female fastener material prior to starting the test. This means the fastening system has a high risk of failing to keep the diaper closed during wearing for consumers who apply the diaper tightly and only lightly press the male fastener to engage it to the female fastener.

### Example 7

The fastener system of Example 7 includes a male fastener that is known in the industry as Velcro 788 and a female fastener integral to the backsheet. The female fastener material is a 15 gsm spunbond nonwoven made of polypropylene having average fiber diameter of 1.8 denier, manufactured by Guelsan Sentetik. The spunbond 15 gsm was thermally bonded having a bond pattern, as shown in FIG. 28. VPT-S testing as detailed above was performed on the fastener system of Example 7. The resulting contour plot is depicted as FIG. 31. The contour plot illustrates that the fastening system requires at least about 0.5 N to be disengaged in the direction perpendicular to the surface for the majority of the test condition space (e.g., range of application force and shear force applied) other than the space above about 5 N shear and less than about 0.2 N application force, in which the system requires between about 0 and about 0.5 N. Many wearing conditions can provide a normal force above this level, so higher than typical failure rates (diaper becoming unfastened during wearing) may occur. Further, in this high shear, low application force portion of the space tested, all samples resulted in the sled moving laterally such that the entire male fastener area is not on the female fastener material prior to starting the test. This means the fastening system has a high risk of failing to keep the diaper closed during wearing for consumers who apply the diaper tightly and only lightly press the male fastener to engage it to the female fastener.

The present disclosure is particularly suitable for use as a primary fastening system of an absorbent article, such as a diaper. In context of a primary fastening system for a diaper, the male fastening member is intended to be fastened to the female fastening member in front waist region 5 of the article and may be fastened adjacent front waist edge 10 and may further be fastened between side edge 3 and side edge 4. In particularly preferred embodiments a primary fastening zone may be designated as the preferred location the article's manufacturer intends for the article to be fastened. The primary fastening zone may be located in the front waist region and be between 30 and 100 in length (measured parallel to the longitudinal axis 80), or between about 35 and 65 mm in length, or between about 40 and 55 mm in length. The primary fastening zone may have a primary fastening zone upper edge and a primary fastening zone lower edge. The primary zone upper edge may be located at the front waist edge 10 or may be longitudinally away from front waist edge 10 and thus toward the longitudinal midpoint by between about 5 and 40 mm, or between about 10 and 25 mm. The primary fastening zone may be laterally centered about the longitudinal axis 80 and may have a width of at least about 75 mm, at least about 100 mm, or at least about 125 mm.

The primary fastening zone includes a female fastener detailed in the present disclosure. The primary fastening zone may also include indicia, such as graphics or other means known in the art (such as a texture) to highlight to the user that it is the intended location for primary fastening.

Fastening systems that comprise the male fastener and the female fastening material that is integral to the outer cover non-woven of the backsheet as detailed above in Example 4 can provide a system with sufficient performance in use. If other male fasteners are utilized in the fastening system, performance is decreased. If other female fastening materials that are integral to the outer cover non-woven of the backsheet are utilized in the fastening system, performance is also decreased. An unexpected positive result has been found between the particular male fastener and the female fastening material that is integral to the outer cover non-woven of the backsheet as detailed above in Example 4.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any embodiment disclosed or claimed herein or that it alone, or in any combination with any other reference or references, teaches, suggests or discloses any such embodiment. Further, to the extent that any meaning or definition of a term in this document conflicts with any meaning or definition of the same term in a document incorporated by reference, the meaning or definition assigned to that term in this document shall govern.

While particular embodiments of the present disclosure have been illustrated and described, it would be obvious to those skilled in the art that various other changes and modifications may be made without departing from the spirit and scope of the present disclosure. It is therefore intended to cover in the appended claims all such changes and modifications that are within the scope of this disclosure.

What is claimed is:

1. An absorbent article comprising:
a) a chassis having i) a topsheet, ii) a backsheet comprising an outer cover non-woven material, and iii) an absorbent core disposed between the topsheet and the backsheet; and
b) a fastening system, the fastening system having a male fastener and a female fastening material,
wherein the male fastener is joined to the at least one elastically elongatable panel, and the male fastener includes a plurality of bidirectional micro-sized hooks, wherein:
each of the hooks has an overall hook height that is greater than or equal to one third of the overall thickness of the female fastening material but less than or equal to twice the overall thickness of the female fastening material;
each of the hooks includes a cap, and each of the caps has a vertical engagement effective area that:
has an overall shape with at least two sides that are substantially parallel, has a width-to-thickness aspect ratio that is greater than or equal to 1 but less than or equal to 2, and
is greater than or equal to 40,000 square micrometers but less than or equal to 120,000 square micrometers; and
each of the caps has an overall cap height that is:
less than or equal to one third of the thickness of the overall thickness of the female fastening material, and
less than or equal to 120 micrometers; and
each of the hooks has two arms, at least one of the arms having an underside, the underside including a recessed portion, the recessed portion having a deepest recessed depth that is greater than or equal to 40% of the overall fiber cross-sectional dimension and less than or equal to 200% of the overall fiber cross-sectional dimension; and
wherein the female fastening material is integral with the outer cover non-woven and the female fastening material comprises a section of a nonwoven web forming surface approximating a plane, a machine direction and a cross direction perpendicular to the machine direction, the nonwoven web formed predominately of polymeric fibers and comprising a series of one or more consolidating bonds impressed on the surface, the one or more consolidating bonds having at least one bond shape; wherein the series is repeated to form a pattern of consolidating bonds; wherein the series is repeated in at least four rows extending predominately in the cross direction, and the series is repeated in at least four columns extending predominately in the machine direction; and wherein the bond shape has a perimeter with a greatest measurable length and a greatest measurable width, and the perimeter:
has a convex portion;
has an aspect ratio of the greatest measurable length to the greatest measurable width of at least 2.5; and
is oriented such that a line intersecting the perimeter along which the greatest measurable length exists intersects an axis lying on the surface along the machine direction to form an angle of between 1 degree and 40 degrees.

2. The absorbent article of claim 1, wherein each of the arms has an underside with a recessed portion.

3. The absorbent article of claim 1, wherein the two sides that are substantially parallel are two side edges of the cap.

4. The absorbent article of claim 1, wherein the overall shape of the vertical engagement effective area is rectangular.

5. The absorbent article of claim 1, wherein the overall hook height is greater than or equal to half of the overall thickness of the female fastening material but less than or equal to one and half times the overall thickness of the female fastening material.

6. The absorbent article of claim 1, wherein the overall hook height is about equal to the overall thickness of the female fastening material.

7. The absorbent article of claim 1, wherein the width-to-thickness aspect ratio is greater than or equal to 1.2, but less than or equal to 1.9.

8. The absorbent article of claim 1, wherein the width-to-thickness aspect ratio is greater than or equal to 1.3, but less than or equal to 1.8.

9. The absorbent article of claim 1, wherein the vertical engagement effective area is greater than or equal to 50,000 square micrometers, but less than or equal to 100,000 square micrometers.

10. The absorbent article of claim 1 wherein the pattern is arranged such that an airflow line may be identified on the surface, that passes at least 8 rows of consolidating bonds, without intersecting a bond shape, and intersects an axis lying on the surface along the cross direction, to form an angle of greater than 45 degrees.

11. The absorbent article of claim 1 wherein the convex portion has varying radii.

12. The absorbent article of claim 1 wherein the perimeter also has a concave portion disposed substantially opposite the convex portion.

13. The absorbent article of claim 1 wherein bonds having the repeated bond shape are arranged in identical rotational orientation along a line lying on the surface along the machine direction.

14. The absorbent article of claim 1 wherein bonds having the repeated bond shape are arranged in identical rotational orientation in a column along a line lying on the surface that intersects an axis lying on the surface along the machine direction to form an angle of less than 4 degrees.

15. The absorbent article of claim 1 wherein the greatest measurable length lies along a line lying on the surface that intersects an axis lying on the surface along the machine direction to form an angle between 5 degrees and 15 degrees.

16. The absorbent article of claim 1 wherein the convex portion has a camber height, and the ratio of the camber height to the greatest measurable length is 0.30 or less.

17. The absorbent article of claim 1 wherein the perimeter has two convex portions.

18. The absorbent article of claim 17 wherein the perimeter has two concave portions respectively disposed substantially opposite the convex portions.

19. The absorbent article of claim 1, wherein when the fastening system is tested under the VPT-S Method, zero out of ten samples fail at the 0.1 N application force, 7 N shear force conditions.

20. The absorbent article of claim 1, wherein the female fastening material has a basis weight of 13 gsm or less.

21. The absorbent article of claim 1, wherein the female fastening material has a basis weight between about 13 gsm and about 25 gsm.

22. The absorbent article of claim 1, wherein the female fastening material has a basis weight between about 10 gsm and about 22 gsm.

23. The absorbent article of claim 1, wherein the female fastening material contains over 50% of fibers made by spunbond process measured by weight.

24. The absorbent article of claim 1, wherein a zone on a front side of the disposable absorbent article can be identified wherein:
a. contains at least a portion of the female fastener, and
b. area size is greater than the area size of one male fastener used in the fastening system, and
c. average adhesive add-on level measured in gram per square meter applied in between the female fastener material and a substantially fluid impermeable component of the backsheet is at least 10% higher than a same size area outside of primary fastening zone.
